(19) United States
(12) Patent Application Publication
(10) Pub. No.: US 2026/0348965 A1
(43) Pub. Date: Nov. 5, 2026

(54) METHODS AND MATERIALS FOR ASSESSING AND TREATING CANCER

(71) Applicant: Mayo Foundation for Medical Education and Research, Rochester, MN (US)

(72) Inventor: Ian F. Parney, Rochester, MN (US)

(21) Appl. No.: 19/488,359

(22) PCT Filed: May 31, 2024
(86) PCT No.: PCT/US2024/032128

§ 371 Date: Nov. 26, 2025

Related U.S. Application Data
(60) Provisional application No. 63/470,058, filed on May 31, 2023.

(51) Int. Cl. G01N 33/575; A61K 31/495; A61P 35/00

(52) U.S. Cl. CPC G01N 33/57557; A61K 31/495; A61P 35/00; G01N 2333/70553; G01N 2333/70589; G01N 2333/70596

(57) ABSTRACT

This document relates to methods and materials for assessing and/or treating mammals (e.g., humans) having brain cancer (e.g., glioblastoma (GBM)). For example, the presence of a population of CD9⁺ extracellular vesicles (EVs; e.g., CD9⁺ plasma EVs) having a distinct profile in a sample (e.g., a blood sample) obtained from a mammal (e.g., a human) can be used to identify that mammal as having brain cancer (e.g., GBM). Also provided are materials and methods for treating mammals (e.g., a human) having brain cancer (e.g., GBM)

### CROSS-REFERENCE TO RELATED APPLICATIONS

This application claims the benefit of U.S. Patent Application Ser. No. 63/470,058, filed on May 31, 2023. The disclosure of the prior application is considered part of (and is incorporated by reference in) the disclosure of this application.

### TECHNICAL FIELD

This document relates to methods and materials for assessing and/or treating mammals (e.g., humans) having brain cancer (e.g., glioblastoma (GBM)). For example, the presence of a population of CD9⁺ extracellular vesicles (EVs; e.g., CD9⁺ plasma EVs) having a distinct profile in a sample (e.g., a blood sample) obtained from a mammal (e.g., a human) can be used to identify that mammal as having brain cancer (e.g., GBM). Also provided are materials and methods for treating mammals (e.g., a human) having brain cancer (e.g., GBM).

### BACKGROUND INFORMATION

GBM (WHO grade IV) is the most common malignant brain tumor in adults (Ostrom et al., Neuro. Oncol., 21:v1-v100 (2019); and Ostrom et al., Prog. Neurol. Surg., 30:1-11 (2018)). Even with maximal safe resection, radiotherapy and adjuvant chemotherapy (Nam et al., J. Oncol. Pract., 13:629-638 (2017)), median overall survival is just over 15 months (Marenco-Hillembrand et al., J. Neurooncol., 147:297-307 (2020)), and 5-year survival is only 4% (Stupp et al., N. Engl. J. Med., 352:987-996 (2005)). Initial diagnosis and subsequent treatment response assessment is generally based on serial clinical evaluation and magnetic resonance imaging (MRI). Unfortunately, MRI findings are non-specific. For example, definitive differentiation between primary malignant brain tumors and secondary metastatic brain tumors is not possible based on MRI alone. Similarly, treatment-related inflammatory pseudoprogression in GBM patients with associated clinical symptoms, new contrast enhancement, and edema indistinguishable from true progression is common (Balana et al., Cancer Med-US, 6:2858-2866 (2017); and Thust et al., J. Magn. Reson. Imaging, 48:571-589 (2018)). Differentiating pseudoprogression from true tumor progression is critical to ensure appropriate management decisions but challenging based on clinical findings and MRI alone. Definitive diagnosis can be made via brain biopsy, but this is invasive, carries risk, and may not capture tumor/necrosis heterogeneity (Muller Bark et al., Br. J. Cancer, 122:295-305 (2020)). Minimally invasive diagnostic tools to augment clinical and imaging findings in GBM patients are a vital, unmet clinical need.

### SUMMARY

This document provides methods and materials for assessing and/or treating mammals (e.g., humans) having brain cancer (e.g., GBM). For example, this document provides methods and materials for identifying a mammal (e.g., a human) as having brain cancer (e.g., GBM) based, at least in part, on the characterization of CD9′ EVs (e.g., CD9′ plasma EVs) in a sample (e.g., a blood sample) obtained from the mammal. In some cases, a sample (e.g., a blood sample) obtained from a mammal can be assessed to identify the mammal as having brain cancer (e.g., GBM) based, at least in part, on the molecular signature of CD9⁺ EVs (e.g., CD9⁺ plasma EVs) in the sample. In some cases, a sample (e.g., a blood sample) obtained from a mammal (e.g., a human) can be assessed to identify the mammal as having brain cancer (e.g., GBM) based, at least in part, on the size of CD9⁺ EVs (e.g., CD9⁺ plasma EVs) in the sample to determine whether the mammal has brain cancer (e.g., GBM). As demonstrated herein, distinct molecular signatures can be associated with plasma EVs in mammals (e.g., human) having brain cancer (e.g., GBM). For example, an altered (e.g., increased or decreased) percentage of the CD9⁺ EVs (e.g., CD9⁺ plasma EVs) from GBM patients can contain (e.g., on a surface of the vesicle) one or more of a CD63 polypeptide, a CD81 polypeptide, a CD11b polypeptide, a CD41a polypeptide, a CD31 polypeptide, and a CD45 polypeptide. For example, an altered (e.g., increased or decreased) concentration of the CD9⁺ EVs (e.g., CD9⁺ plasma EVs) from GBM patients can contain (e.g., on a surface of the vesicle) one or more of a CD63 polypeptide, a CD81 polypeptide, a CD11b polypeptide, a CD41a polypeptide, a CD31 polypeptide, and a CD45 polypeptide. For example, an altered (e.g., increased or decreased) intensity (e.g., an altered mean fluorescence intensity (MFI)) of the CD9⁺ EVs (e.g., CD9⁺ plasma EVs) from GBM patients can contain (e.g., on a surface of the vesicle) one or more of a CD63 polypeptide, a CD81 polypeptide, a CD11b polypeptide, a CD41a polypeptide, a CD31 polypeptide, and a CD45 polypeptide. Also as demonstrated herein, CD9 EVs (e.g., CD9⁺ plasma EVs) from GBM patients can be a different (e.g., a larger or a smaller) size than CD9⁺ EVs from patients who do not have GBM (e.g., as measured by light side scatter). For example, CD9⁺ plasma EVs from GBM patients that contain (e.g., on a surface of the vesicle) a CD81 polypeptide can have an average longest dimension (e.g., an average diameter) of less than about 750 nanometers (nm). For example, CD9⁺ plasma EVs from GBM patients that contain (e.g., on a surface of the vesicle) a CD63 polypeptide and a CD81 polypeptide can have an average longest dimension (e.g., an average diameter) of less than about 1400 nm. For example, CD9⁺ plasma EVs from GBM patients that contain (e.g., on a surface of the vesicle) a CD45 polypeptide can have an average longest dimension (e.g., an average diameter) of greater than about 1000. The ability to use a simple, non-invasive liquid biopsy technique to identify a mammal (e.g., a human) as having a brain cancer such as GBM as described herein (e.g., based, at least in part, on the molecular signature and/or size of CD9⁺ EVs (e.g., CD9⁺ plasma EVs) in a sample obtained from the mammal) provides a unique and unrealized opportunity for early detection of GBM, early treatment of GBM, and the ability to monitor GBM treatment responses. Non-invasive techniques for detecting GMB also can spare patients from unnecessary biopsies and/or poorly informed treatment decisions.

In general, one aspect of this document features methods for identifying a mammal as having a brain cancer. The methods can include, or consist essentially of, (a) determining a percentage of CD9⁺ EVs in a blood sample obtained from a mammal that contain a CD81 polypeptide, and (b) classifying the mammal as having the brain cancer if greater than 1 percent of the CD9⁺ EVs contain the CD81 polypeptide; and (c) classifying the mammal as not having the brain cancer if less than 1 percent of the CD9+ EVs contain the CD81 polypeptide. The mammal can be a human. The brain cancer can be a glioma (e.g., a GBM), an acoustic neuroma, a pituitary adenoma, or a medulloblastoma. The blood sample can be a whole blood sample, a plasma sample, or a serum sample. The method can include determining that greater than 1 percent of the CD9⁺ EVs contain the CD81 polypeptide, and classifying the mammal as having the brain cancer. The method can include determining that greater than 1 percent of the CD9⁺ EVs contain the CD81 polypeptide, and classifying the brain cancer as a glioblastoma. The method can include determining that less than 1 percent of the CD9⁺ EVs contain the CD81 polypeptide, and classifying the mammal as not having the brain cancer.

In another aspect, this document features methods for treating brain cancer. The methods can include, or consist essentially of, (a) determining that greater than 1 percent of CD9⁺ EVs in a blood sample obtained from a mammal suspected of having brain cancer contain a CD81 polypeptide, and (b) administering a brain cancer treatment to the mammal. The mammal can be a human. The brain cancer can be a glioma (e.g., a GBM), an acoustic neuroma, a pituitary adenoma, or a medulloblastoma. The blood sample can be a whole blood sample, a plasma sample, or a serum sample. The brain cancer treatment can include subjecting the mammal to radiation therapy. The brain cancer treatment can include administering temozolomide to the mammal. The brain cancer treatment can include subjecting the mammal to radiation therapy and administering temozolomide to the mammal.

In another aspect, this document features methods for treating brain cancer. The methods can include, or consist essentially of, administering a brain cancer treatment to a mammal identified as having a blood sample including greater than 1% of CD9⁺ EVs in the blood sample that contain a CD81 polypeptide. The mammal can be a human. The brain cancer can be a glioma (e.g., a GBM), an acoustic neuroma, a pituitary adenoma, or a medulloblastoma. The blood sample can be a whole blood sample, a plasma sample, and a serum sample. The brain cancer treatment can include subjecting the mammal to radiation therapy. The brain cancer treatment can include administering temozolomide to the mammal. The brain cancer treatment can include subjecting the mammal to radiation therapy and administering temozolomide to the mammal.

In another aspect, this document features methods for identifying a mammal as having a brain cancer. The methods can include, or consist essentially of, (a) determining a size of CD9⁺ EVs that contain a CD81 polypeptide in a blood sample obtained from the mammal, and (b) classifying the mammal as having the brain cancer if the CD9⁺ EVs containing the CD81 polypeptide have a size of less than 750 nanometers (nm); or (c) classifying the mammal as not having the brain cancer the CD9⁺ EVs containing the CD81 polypeptide have a size of greater than 750 nm. The mammal can be a human. The brain cancer can be a glioma, an acoustic neuroma, a pituitary adenoma, or a medulloblastoma.

The blood sample can be a whole blood sample, a plasma sample, or a serum sample. The CD9⁺ EVs containing the CD81 polypeptide can have a size of less than 750 nm, and the method can include classifying the mammal as having the brain cancer. The CD9⁺ EVs containing the CD81 polypeptide can have a size of greater than 750 nm, and the method can include classifying the mammal as not having the brain cancer.

In another aspect, this document features methods for treating brain cancer. The methods can include, or consist essentially of, (a) determining that CD9⁺ EVs in a blood sample obtained from a mammal suspected of having brain cancer contain a CD81 polypeptide and have a size of less than 750 nm, and (b) administering a brain cancer treatment to the mammal. The mammal can be a human. The brain cancer can be a glioma, an acoustic neuroma, a pituitary adenoma, or a medulloblastoma. The blood sample can be a whole blood sample, a plasma sample, or a serum sample. The brain cancer treatment can include subjecting the mammal to radiation therapy. The brain cancer treatment can include administering temozolomide to the mammal. The brain cancer treatment can include subjecting the mammal to radiation therapy and administering temozolomide to the mammal.

In another aspect, this document features methods for treating brain cancer. The methods can include, or consist essentially of, administering a brain cancer treatment to a mammal identified as having a blood sample including CD9⁺ EVs that contain a CD81 polypeptide and have a size of less than 750 nm. The mammal can be a human. The brain cancer can be a glioma, an acoustic neuroma, a pituitary adenoma, or a medulloblastoma. The blood sample can be a whole blood sample, a plasma sample, or a serum sample. The brain cancer treatment can include subjecting the mammal to radiation therapy. The brain cancer treatment can include administering temozolomide to the mammal. The brain cancer treatment can include subjecting the mammal to radiation therapy and administering temozolomide to the mammal.

In another aspect, this document features methods for identifying a mammal as having a brain cancer. The methods can include, or consist essentially of, (a) determining a percentage of CD9⁺ EVs in a blood sample obtained from the mammal that contain a CD11b polypeptide, and (b) classifying the mammal as having the brain cancer if greater than 5 percent of the CD9⁺ EVs contain the CD11b polypeptide; or (c) classifying the mammal as not having the brain cancer if less than 5 percent of the CD9⁺ EVs contain the CD11b polypeptide. The mammal can be a human. The brain cancer can be a glioma, an acoustic neuroma, a pituitary adenoma, or a medulloblastoma. The blood sample can be a whole blood sample, a plasma sample, or a serum sample. The method can include determining that greater than 5 percent of the CD9⁺ EVs contain the CD11b polypeptide, and classifying the mammal as having the brain cancer. The method can include determining that less than 5 percent of the CD9⁺ EVs contain the CD11b polypeptide, and classifying the mammal as not having the brain cancer.

In another aspect, this document features methods for treating brain cancer. The methods can include, or consist essentially of, (a) determining that greater than 5 percent of CD9⁺ EVs in a blood sample obtained from a mammal suspected of having brain cancer contain a CD11b polypeptide, and (b) administering a brain cancer treatment to the mammal. The mammal can be a human. The brain cancer can be a glioma, an acoustic neuroma, a pituitary adenoma, or a medulloblastoma. The blood sample can be a whole blood sample, a plasma sample, or a serum sample. The brain cancer treatment can include subjecting the mammal to radiation therapy. The brain cancer treatment can include administering temozolomide to the mammal. The brain cancer treatment can include subjecting the mammal to radiation therapy and administering temozolomide to the mammal.

In another aspect, this document features methods for treating brain cancer. The methods can include, or consist essentially of, administering a brain cancer treatment to a mammal identified as having a blood sample including greater than 5% of CD9⁺ EVs in the blood sample that contain a CD11b polypeptide. The mammal can be a human. The brain cancer can be a glioma, an acoustic neuroma, a pituitary adenoma, or a medulloblastoma. The blood sample can be a whole blood sample, a plasma sample, or a serum sample. The brain cancer treatment can include subjecting the mammal to radiation therapy. The brain cancer treatment can include administering temozolomide to the mammal. The brain cancer treatment can include subjecting the mammal to radiation therapy and administering temozolomide to the mammal.

In another aspect, this document features methods for identifying a mammal as having a brain cancer. The methods can include, or consist essentially of, (a) determining a size of CD9⁺ EVs containing a CD63 polypeptide and a CD81 polypeptide in a blood sample obtained from the mammal, and (b) classifying the mammal as having the brain cancer if the CD9⁺ EVs containing the CD63 polypeptide and the CD81 polypeptide have a size of less than 1400 nm; or (c) classifying the mammal as not having the brain cancer if the CD9⁺ EVs containing the CD63 polypeptide and the CD81 polypeptide have a size of greater than 1400 nm. The mammal can be a human. The brain cancer can be a glioma, an acoustic neuroma, a pituitary adenoma, or a medulloblastoma. The blood sample can be a whole blood sample, a plasma sample, or a serum sample. The CD9⁺ EVs containing the CD63 polypeptide and the CD81 polypeptide can have a size of less than 1400 nm, and the method can include classifying the mammal as having the brain cancer. The CD9⁺ EVs containing the CD63 polypeptide and the CD81 polypeptide can have a size of greater than 1400 nm, and the method can include classifying the mammal as not having the brain cancer.

In another aspect, this document features methods for treating brain cancer. The methods can include, or consist essentially of, (a) determining that CD9⁺ EVs containing a CD63 polypeptide and a CD81 polypeptide in a blood sample obtained from a mammal suspected of having brain cancer have a size of less than 1400 nm, and (b) administering a brain cancer treatment to the mammal. The mammal can be a human. The brain cancer can be a glioma, an acoustic neuroma, a pituitary adenoma, or a medulloblastoma. The blood sample can be a whole blood sample, a plasma sample, or a serum sample. The brain cancer treatment can include subjecting the mammal to radiation therapy. The brain cancer treatment can include administering temozolomide to the mammal. The brain cancer treatment can include subjecting the mammal to radiation therapy and administering temozolomide to the mammal.

In another aspect, this document features methods for treating brain cancer. The methods can include, or consist essentially of, administering a brain cancer treatment to a mammal identified as having a blood sample including CD9⁺ EVs that contain a CD63 polypeptide and a CD81 polypeptide and have a size of less than 1400 nm. The mammal can be a human. The brain cancer can be a glioma, an acoustic neuroma, a pituitary adenoma, or a medulloblastoma. The blood sample can be a whole blood sample, a plasma sample, or a serum sample. The brain cancer treatment can include subjecting the mammal to radiation therapy. The brain cancer treatment can include administering temozolomide to the mammal. The brain cancer treatment can include subjecting the mammal to radiation therapy and administering temozolomide to the mammal.

In another aspect, this document features methods for identifying a mammal as having a brain cancer. The methods can include, or consist essentially of, (a) determining a concentration of CD9⁺ EVs in a blood sample obtained from the mammal that contain a CD11b polypeptide, and (b) classifying the mammal as having the brain cancer if the CD9⁺ EVs containing the CD11b polypeptide have a concentration of greater than 500 events/microliter (μL); or (c) classifying the mammal as not having the brain cancer the CD9⁺ EVs containing the CD11b polypeptide have a concentration of less than 500 events/μL. The mammal can be a human. The brain cancer can be a glioma, an acoustic neuroma, a pituitary adenoma, or a medulloblastoma. The blood sample can be a whole blood sample, a plasma sample, or a serum sample. The CD9⁺ EVs containing the CD11b polypeptide can have a concentration of greater than 500 events/μL, and the method can include classifying the mammal as having the brain cancer. The CD9⁺ EVs containing the CD11b polypeptide can have a concentration of less than 500 events/μL, and the method can include classifying the mammal as not having the brain cancer.

In another aspect, this document features methods for treating brain cancer. The methods can include, or consist essentially of, (a) determining that CD9⁺ EVs containing a CD11b polypeptide in a blood sample obtained from a mammal suspected of having brain cancer have a concentration of greater than 500 events/μL, and (b) administering a brain cancer treatment to the mammal. The mammal can be a human. The brain cancer can be a glioma, an acoustic neuroma, a pituitary adenoma, or a medulloblastoma. The blood sample can be a whole blood sample, a plasma sample, or a serum sample. The brain cancer treatment can include subjecting the mammal to radiation therapy. The brain cancer treatment can include administering temozolomide to the mammal. The brain cancer treatment can include subjecting the mammal to radiation therapy and administering temozolomide to the mammal.

In another aspect, this document features methods for treating brain cancer. The methods can include, or consist essentially of, administering a brain cancer treatment to a mammal identified as having a blood sample including a concentration of CD9⁺ EVs containing a CD11b polypeptide of greater than 500 events/μL. The mammal can be a human. The brain cancer can be a glioma, an acoustic neuroma, a pituitary adenoma, or a medulloblastoma. The blood sample can be a whole blood sample, a plasma sample, or a serum sample. The brain cancer treatment can include subjecting the mammal to radiation therapy. The brain cancer treatment can include administering temozolomide to the mammal. The brain cancer treatment can include subjecting the mammal to radiation therapy and administering temozolomide to the mammal.

In another aspect, this document features methods for identifying a mammal as having a brain cancer. The methods can include, or consist essentially of, (a) determining a size of CD9⁺ EVs containing a CD45 polypeptide in a blood sample obtained from the mammal, and (b) classifying the mammal as having the brain cancer if the CD9⁺ EVs containing the CD45 polypeptide have a size of greater than 1000 nm; or (c) classifying the mammal as not having the brain cancer if the CD9⁺ EVs containing the CD45 polypeptide have a size of less than 1000 nm. The mammal can be a human. The brain cancer can be a glioma, an acoustic neuroma, a pituitary adenoma, or a medulloblastoma. The blood sample can be a whole blood sample, a plasma sample, or a serum sample. The CD9⁺ EVs containing the CD45 polypeptide can have a size of greater than 1000 nm, and the method can include classifying the mammal as having the brain cancer. The CD9⁺ EVs containing the CD45 polypeptide can have a size of less than 1000 nm, and the method can include classifying the mammal as not having the brain cancer.

In another aspect, this document features methods for treating brain cancer. The methods can include, or consist essentially of, (a) determining that CD9⁺ EVs containing a CD45 polypeptide in a blood sample obtained from a mammal suspected of having brain cancer have a size of greater than 1000 nm, and (b) administering a brain cancer treatment to the mammal. The mammal can be a human. The brain cancer can be a glioma, an acoustic neuroma, a pituitary adenoma, or a medulloblastoma. The blood sample can be a whole blood sample, a plasma sample, or a serum sample. The brain cancer treatment can include subjecting the mammal to radiation therapy. The brain cancer treatment can include administering temozolomide to the mammal. The brain cancer treatment can include subjecting the mammal to radiation therapy and administering temozolomide to the mammal.

In another aspect, this document features methods for treating brain cancer. The methods can include, or consist essentially of, administering a brain cancer treatment to a mammal identified as having a blood sample including CD9⁺ EVs containing a CD45 polypeptide and having a size of greater than 1000 nm. The mammal can be a human. The brain cancer can be a glioma, an acoustic neuroma, a pituitary adenoma, or a medulloblastoma. The blood sample can be a whole blood sample, a plasma sample, or a serum sample. The brain cancer treatment can include subjecting the mammal to radiation therapy. The brain cancer treatment can include administering temozolomide to the mammal. The brain cancer treatment can include subjecting the mammal to radiation therapy and administering temozolomide to the mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-IF show human plasma processing and EV staining assay controls. FIG. 1A) A schematic showing an exemplary method for plasma isolation from whole blood. FIG. 1B) Cytek Aurora Flow cytometer settings were optimized to visualize different sizes of fluorescent and non-fluorescent ApogeeMix beads. FIG. 1C) Serial dilution demonstrated a linear relationship between dilution and detected events for fluorescent beads (110 nm) and non-fluorescent beads (880 nm), suggesting clumping was absent. FIG. 1D) EVs isolated from CD14⁺ monocytes in vitro were stained with CD9 (upper panel) and CD11b (lower panel). FIG. 1E) EVs isolated from CD14⁺ monocytes in vitro were stained with CD9/CD11b together. In FIGS. 1D-1E, controls included buffer-only (PBS), buffer with reagent (PBS+Ab), single color stained EVs (EV+Ab), and stained but detergent-treated EV samples (EV+Ab+SDS). FIG. 1F) Serial dilution of CD9-stained EVs versus detected events. A linear relationship between dilution and detected events was observed.

FIGS. 2A-2B compare methods to separate and concentrate stained EVs from unbound antibodies. FIG. 2A) Representative dot plots for CD9-stained EVs from CD14⁺ monocytes in vitro versus side scatter (SSC)-A without removing unbound antibodies compared to after differential ultracentrifugation (DU), ultrafiltration, size exclusion chromatography (SEC), or density gradient ultracentrifugation (DGU) to eliminate excess antibodies. PBS+CD9 antibodies were used as a process control (lower panel). FIG. 2B) All four methods reduced EV yield, but SEC was most effective at separating/concentrating EVs with the highest signal-to-noise ratio.

FIGS. 3A-3E show an EV staining panel, assay controls, and data acquisition for plasma EV analysis. FIG. 3A) A schematic outlining exemplary staining and separation/concentration methods for plasma EVs. FIGS. 2B and 2C) CD9 antibody titration for plasma EVs samples did not show significant differences in event rates (FIG. 3B) or mean fluorescence intensity (MFI, FIG. 3C) with increasing amounts of CD9 antibodies. FIG. 3D) Representative dot plots showing the percentage of positive events for plasma EV surface marker expression determined using an SSC threshold for EV-associated tetraspanins (CD9, CD63, and CD81), phalloidin (staining actin as a marker of contaminating cell debris), and leukocyte/platelet/endothelial markers (CD11b, CD31, CD41a, and CD45). Assays controls included buffer only (PBS), buffer with reagents (PBS+all antibodies), single color staining, all color staining, and fluorescence minus one (FMO; all antibodies except one directed against the marker of interest). FIG. 3E) Changing from a size-based SSC threshold for event acquisition (upper panel) to a spectral fluorescence based surface marker expression threshold (lower panel) reduced non-specific background staining as demonstrated for CD9 expression in SEC-purified stained EVs. ns=not significant.

FIGS. 4A-4J show an analysis of plasma EVs in samples from GBM and normal donors. FIG. 4A) Dot plots showing an exemplary gating strategy and controls for particles in the EV size range (EV region). FIG. 4B) Pooled data from 20 patients diagnosed with GBM and 20 normal donors (ND) showing percentage of EV-associated tetraspanin (CD9, CD63, and CD81) and cell of origin (CD45, CD11b, CD31, and CD41a) surface marker expression among EV region/phalloidin-negative particles. CD9⁺ and CD41a⁺ EVs were most abundant. Tetraspanin expression was more common in samples from GBM patients than in samples from ND. FIG. 4C) Most plasma EVs expressed CD9 tetraspanin only. GBM patients had increased CD9⁺, CD9⁺/CD81*, and CD9⁺/CD63⁺/CD81⁻ EVs. FIGS. 4D-4G) Most CD45%, CD11b, CD31⁺/CD41a⁺, and CD31⁺/CD41a⁻ EV populations in ND only expressed the tetraspanin CD9 while GBM patients had increased frequencies of CD9⁺, CD9⁺/CD81⁺, and CD9⁺/CD63⁺/CD81⁻ EVs. FIG. 4H) Strategy for tracing cell of origin for CD9⁺/Phalloidin EVs. FIG. 4I) No statistically significant differences between EVs in samples from GBM patients and ND for CD45⁺ (leukocyte common antigen) or CD31⁺CD41a (endothelial) plasma EVs. Samples from GBM patients had increased CD11b⁻/CD45⁻ EVs (presumed myeloid origin) while samples from ND had increased CD31⁺CD41a⁺ (platelet-derived) EVs. FIG. 4J) Most CD11b⁺ plasma EVs did not express CD45. This was distinct from myeloid cells, which would typically be CD11b⁺/CD45⁺. Statistical significance is indicated with asterisks. *p<0.05, **p<0.01, ***p<0.001, ****p <0.0001.

FIGS. 5A-5C show that T-SNE and FlowSOM analyses revealed differently expressed plasma EV subpopulations in GBM and normal donors. FIG. 5A) T-SNE (t-distributed stochastic neighbor embedding) analysis based on SSC, CD9, CD81, CD63, CD31, CD45, CD11b, and CD41a expression and shows different plasma EV clustering features for samples from GBM patients and normal donors. FIG. 5B) FlowSOM (flow cytometry self-organizing map) analysis and shows 15 EV subpopulations differing in size (SSC) and surface marker expression. FIG. 5C) Some groups are enriched in samples from ND (Pop0, Pop2, Pop6) while groups are enriched in samples from GBM patients (Pop1, Pop3, Pop7). Statistical significance is indicated with asterisks *p<0.05, ***p<0.001, ****p<0.0001.

FIG. 6 is a series of plots of fluorescence versus SCC-A. It shows the impact of spectral flow cytometer thresholds on visualizing nanoparticle beads. After optimizing data collection settings, an SSC-500 threshold visualized both fluorescent and non-fluorescent beads of varying sizes (upper left panel). Changing the threshold to fluorescence intensity triggering with various spectral channels led to better fluorescent bead visualization and minimized background noise but did not visualize non-fluorescent beads.

FIG. 7 is plot and accompanying images of nanoparticle bead detection using an ImageStream©XMkII Imaging Flow Cytometer. ApogeeMixed beads were also analyzed with an ImageStream©X MkII Imaging Flow Cytometer. A single clustered population (gated as 80+110 nm) was seen and comprised 2 fluorescent bead populations (1, 2), based on their different sizes in bright field channel (Ch01) and fluorescent intensity (Ch02). However, ImageStream resolution of non-fluorescent beads was inferior compared to spectral flow cytometry with an SSC-500 threshold.

FIG. 8 is a plot showing nanoparticle tracking analysis and protein expression in CD14⁺ monocyte derived EVs in vitro. Nanoparticle tracking analysis (NTA) histogram (left) confirmed the presence of EV-sized particles after differential ultracentrifugation-mediated EV separation and concentration from CD14⁺ monocyte-conditioned media. Western blot (right) demonstrates expression of EV-associated proteins including tetraspanins (CD9, CD63, and CD81) and heat shock proteins (HSP90) as well as a myeloid marker (CD11b). The mean nanoparticle size was 143.2 nm and the concentration was 2.48×10⁹ particles per milliliter.

FIGS. 9A-9C show a comparison of plasma EV single color staining for CD11b, CD31 and CD45 using differently labeled antibodies. The signal-to-noise ratio of positive staining of plasma EVs versus PBS+antibody controls was determined using antibodies directed at the same target but linked to different fluorochromes. Plasma EV staining protocols were standardized for all antibodies. Of the antibodies tested, CD11b PE-Cy7 (FIG. 9A), CD31 APC-Cy7 (FIG. 9B) and CD45 BV510 (FIG. 9C) showed the highest signal-to-noise ratios for each target protein and were used for subsequent multicolor EV staining panel.

FIGS. 10A-10B show poor performance of a lipophilic dye (PKH67) dye as a pan EV marker. Plasma EV staining performed with 1 μm PKH67 alone (FIG. 10A) showed substantial staining above background that was largely eliminated by exposure to detergent (SDS), suggesting EV staining. However, two color staining (FIG. 10B) with PKH67 plus a second antibody directed against cell of origin surface markers (CD11b, CD41a, CD45, or CD31) showed minimal overlapping staining, though detergent (0.2% SDS) eliminated most single positive staining.

FIGS. 11A-11B show separation and concentration of CD11b-stained CD14⁺ monocyte derived EVs. FIG. 11A) Representative dot plots for CD11b-stained EVs from CD14⁺ monocytes in vitro without removing unbound antibodies compared to after DU, ultrafiltration, SEC, or DGU to eliminate excess antibodies. PBS+CD11b antibodies were used as process control (lower panel). FIG. 11B) All four methods reduced EV yield, but SEC was most effective at separating/concentrating EVs with the highest signal-to-noise ratio.

FIG. 12 shows individual size and surface marker expression in GBM and ND plasma samples. EVs dot plots and heat maps showing size (SSC) and surface marker expression in phalloidin-negative plasma EVs from samples from 10 GBM patients and 10 normal donors (ND) with 80000 events per replicate compared to normal donors (NS). GBM patients' plasma EVs are bigger (higher SSC) and have higher expression of CD9, CD81 and CD11b.

FIGS. 13A-13D show results of a comparison of plasma EVs in GBM versus other brain tumors versus normal donors. FIG. 13A) tSNE plots showing distinct expression patterns for GBM versus brain metastases (mets) versus normal donors based on multiparametric CD9, CD63, CD81, CD45, CD11b, CD31, CD41a, and SSC assessment.

FIG. 13B) FLOSOM analysis based on the same tSNE plots demonstrating different expression of different EV populations identified by multiparametric analysis. FIG. 13C) Differential CD9 MFI in GBM, brain mets, normal donors, gliosis/necrosis, and grade 2 glioma. FIG. 13D) Differential CD9 percentage in the same populations.

FIGS. 14A-14B show relative expression of single surface markers by plasma EVs in GBM, brain mets, and grade 2 glioma patient versus normal donors. CD9, CD63, CD81, CD45, CD11 b, CD31, CD41a expression in plasma EVs by percentage (FIG. 14A) and by concentration (FIG. 14B).

FIGS. 15A-15B show relative expression of single surface markers by plasma EVs in GBM, brain mets, and grade 2 glioma patient versus normal donors. CD9, CD63, CD81, CD45, CD11b, CD31, CD41a expression in plasma EVs by size (FIG. 15A) and by MFI (FIG. 15B).

FIGS. 16A-16B show relative expression of combinations of surface markers by plasma EVs in GBM, brain mets, and grade 2 glioma patient versus normal donors. CD9, CD63, CD81, CD45, CD11b, CD31, CD41a expression in plasma EVs by percentage (FIG. 16A) and by concentration (FIG. 16B).

FIG. 17 shows relative expression of combinations of surface markers by plasma EVs in GBM, brain mets, and grade 2 glioma patient versus normal donors. CD9, CD63, CD81, CD45, CD11b, CD31, CD41a expression in plasma EVs by size.

FIGS. 18A-18B show relative expression of combinations of surface markers by CD9+ plasma EVs in GBM, brain mets, and grade 2 glioma patient versus normal donors. CD63, CD81, CD45, CD11b, CD31, CD41a expression in plasma EVs by percentage (FIG. 18A) and by concentration (FIG. 18B).

FIG. 19 shows relative expression of combinations of surface markers by CD9+ plasma EVs in GBM, brain mets, and grade 2 glioma patient versus normal donors. CD63, CD81, CD45, CD11b, CD31, CD41a expression in plasma EVs by size.

FIG. 20 shows an analysis of plasma EVs in samples from GBM and normal donors (ND). Samples from GBM patients had increased CD11b+ EVs (presumed myeloid origin). There was no statistically significant difference between EVs in samples from GBM patients and ND for CD45+ (leukocyte common antigen) EVs. There was no statistically significant difference between EVs in samples from GBM patients and ND for CD45+/CD11b EVs.

FIG. 21 shows the ability of CD9 and CD81 expression to distinguish GBM patients from normal donors (ND). Both CD9 and CD81 plasma EV expression accurately distinguished GBM patients from NDs.

FIGS. 22A-22D show relative expression of combinations of surface markers by plasma EVs (FIG. 22A and FIG. 22B) and CD9+ plasma EVs (FIG. 22C) in pre-operative glioblastoma (GBM Pre-op) and post-operative GBM (GBM Post-op) patients and normal donors (ND). EVs expressing tetraspanins (CD9, CD63, CD81), leukocyte markers (CD45), platelet and endothelial cell markers (CD31, CD41a), and myeloid markers (CD11b) alone or in combination have distinct frequencies (percentage), concentration (particles/μL), size (nm), and mean fluorescence intensity (MFI).

FIGS. 23A-23C show that the frequency of CD9+, CD41a+, CD63+, and CD9+/CD63−/CD81− plasma EVs in glioblastoma (GBM) at diagnosis correlated with both total tumor volume (FIG. 23A) and enhancing tumor volume (FIG. 23B). FIG. 24C is a schematic showing how enhancing tumor volume can be used to guide surgical decision making (FIG. 23C).

FIGS. 24A-24B are survival curves (FIG. 24A) and statistical analyses (FIG. 24B) showing that the frequency of CD9+, CD9+/CD63−/CD81−, and CD9+/CD11b+ plasma EVs in GBM at diagnosis, as well as age, gross total resection and MGMT promoter methylation were independently correlated with overall survival.

FIGS. 25A-25C show an EV tetraspanain data-based machine learning approach used to predict GBM with accuracy.

### DETAILED DESCRIPTION

This document relates to methods and materials for assessing and/or treating mammals (e.g., humans) having brain cancer (e.g., GBM). For example, the presence of a distinct molecular signature in CD9⁺ EVs (e.g., CD9⁺ plasma EVs) in a sample (e.g., a blood sample) obtained from a mammal (e.g., a human) can be used to identify that mammal as having brain cancer (e.g., GBM). In some cases, the size of CD9⁺ EVs (e.g., CD9⁺ plasma EVs) in a sample (e.g., a blood sample) obtained from a mammal (e.g., a human) can be used to identify that mammal as having brain cancer (e.g., GBM). Also provided are materials and methods for treating mammals (e.g., a human) having brain cancer (e.g., GBM).

Any type of mammal can be assessed and/or treated as described herein. Examples of mammals that can have brain cancer (e.g., GBM) and that can be assessed and/or treated as described herein include, without limitation, humans, non-human primates (e.g., monkeys), dogs, cats, horses, cows, pigs, sheep, rabbits, mice, and rats. In some cases, a human can be assessed and/or treated as described herein.

A mammal (e.g., human) having (or suspected of having) any type of brain cancer can be assessed and/or treated as described herein. In some cases, a cancer treated as described herein can include one or more solid tumors. In some cases, a cancer treated as described herein can be a blood cancer. In some cases, a cancer treated as described herein can be a primary cancer. In some cases, a cancer treated as described herein can be a metastatic cancer. In some cases, a cancer treated as described herein can be a refractory cancer. In some cases, a cancer treated as described herein can be a relapsed cancer. Examples of brain cancers that can assessed and/or treated as described herein include, without limitation, gliomas (e.g., GBMs), acoustic neuromas (schwannomas), pituitary adenomas, medulloblastomas, and lymphomas. When a brain cancer is a glioma, the glioma can be any grade glioma (e.g., grade 1 glioma, grade 2 glioma, grade 3 glioma, or grade 4 glioma (GBM)).

Any appropriate EVs can be used in the methods and materials described herein. Examples of EVs include, without limitation, exosomes, microvesicles, and apoptotic bodies. In some cases, an EV can be an exosome.

EVs present in any appropriate type of same sample can be assessed to determine if a mammal (e.g., a human) has brain cancer (e.g., GBM). For example, biological samples such as blood samples (e.g., whole blood samples, serum samples, and plasma samples) can be obtained from a mammal and assessed as described herein. In some cases, a plasma sample can be obtained from a mammal (e.g., a human) and assessed as described herein.

A sample (e.g., a blood sample) that can be assessed as described herein can include any appropriate number of CD9⁺ EVs. For example, a plasma sample including greater than about 1000 CD9⁺ EVs per milliliter of sample (EVs/mL) can be assessed as described herein. In some cases, a sample (e.g., a blood sample) that can be assessed as described herein can include from about 1000 CD9⁺ EVs/mL to about 100000000 CD9⁺ EVs/mL (e.g., from about 1000 to about 10000000, from about 1000 to about 1000000, from about 1000 to about 100000, from about 1000 to about 10000, from about 10000 to about 100000000, from about 100000 to about 100000000, from about 1000000 to about 100000000, from about 10000000 to about 100000000, from about 10000 to about 10000000, from about 100000 to about 1000000, from about 10000 to about 100000, from about 100000 to about 1000000, or from about 1000000 to about 10000000 CD9⁺ EVs/mL).

In some cases, the molecular signature of a population of CD9⁺ EVs (e.g., CD9⁺ plasma EVs) in a sample (e.g., a blood sample) obtained from a mammal (e.g., a human) can be used to identify the mammal as having brain cancer (e.g., GBM). For example, a population of CD9⁺ EVs (e.g., CD9⁺ plasma EVs) in a sample (e.g., a blood sample) obtained from a mammal (e.g., a human) can be assessed to determine the percentage of the CD9⁺ EVs (e.g., CD9⁺ plasma EVs) that contain (e.g., on a surface of the vesicle) one or more (e.g., one, two, three, or more) of a CD63 polypeptide, a CD81 polypeptide, a CD11b polypeptide, a CD41a polypeptide, a CD31 polypeptide, and a CD45 polypeptide, thereby determining whether the mammal has brain cancer (e.g., GBM). In some cases, a population of CD9⁺ EVs (e.g., CD9⁺ plasma EVs) in a sample (e.g., a blood sample) from a mammal (e.g., a human) having brain cancer (e.g., GBM) can have an altered (e.g., increased or decreased) percentage of CD9⁺ EVs that contain (e.g., on a surface of the vesicle) one or more of a CD63 polypeptide, a CD81 polypeptide, a CD11b polypeptide, a CD41a polypeptide, a CD31 polypeptide, and a CD45 polypeptide. The term “increased percentage” as used herein with respect to a percentage of a population of EVs (e.g., a population of CD9⁺ EVs) that contain a particular polypeptide or combination of particular polypeptides (e.g., a CD63 polypeptide, a CD81 polypeptide, a CD11b polypeptide, a CD41a polypeptide, a CD31 polypeptide, and/or a CD45 polypeptide) refers to any percentage that is greater than the percentage of a comparable population of EVs that contain that particular polypeptide or combination of particular polypeptides in a control sample. In some cases, an increased percentage of CD9 EVs that contain (e.g., on a surface of the vesicle) one or more of a CD63 polypeptide, a CD81 polypeptide, a CD11b polypeptide, a CD41a polypeptide, a CD31 polypeptide, and a CD45 polypeptide can be at least 1 order of magnitude (e.g., 1, 2, 3, or more orders of magnitude) greater than the percentage of a comparable population of EVs that contain that particular polypeptide or combination of particular polypeptides in a control sample. The term “decreased percentage” as used herein with respect to a percentage of a population of EVs (e.g., a population of CD9⁺ EVs) that contain a particular polypeptide or combination of particular polypeptides (e.g., a CD63 polypeptide, a CD81 polypeptide, a CD11b polypeptide, a CD41a polypeptide, a CD31 polypeptide, and/or a CD45 polypeptide) refers to any percentage that is less than the percentage of a comparable population of EVs that contain that particular polypeptide or combination of particular polypeptides in a control sample. In some cases, a decreased percentage of CD9⁻ EVs that contain (e.g., on a surface of the vesicle) one or more of a CD63 polypeptide, a CD81 polypeptide, a CD11b polypeptide, a CD41a polypeptide, a CD31 polypeptide, and a CD45 polypeptide can be at least 1 order of magnitude (e.g., 1, 2, 3, or more orders of magnitude) less than the percentage of a comparable population of EVs that contain that particular polypeptide or combination of particular polypeptides in a control sample. Control samples are samples obtained from normal (e.g., healthy) mammals.

In some cases, the molecular signature of a population of CD9⁺ EVs (e.g., CD9⁺ plasma EVs) in a sample (e.g., a blood sample) from a mammal (e.g., a human) having brain cancer (e.g., GBM) can have an altered (e.g., increased or decreased) percentage of the CD9⁺ EVs that contain (e.g., on a surface of the vesicle) at least one of a CD63 polypeptide, a CD81 polypeptide, a CD11b polypeptide, a CD41a polypeptide, a CD31 polypeptide, and a CD45 polypeptide.

In some cases, when a sample (e.g., a plasma sample) obtained from a mammal (e.g., a human) suspected of having cancer (e.g., brain cancer) is determined to have at least 1 percent of the CD9⁺ EVs (e.g., CD9⁺ plasma EVs) in that sample containing a CD81 polypeptide, then that mammal can be identified as having a brain cancer (e.g., GBM). For example, when a sample (e.g., a plasma sample) obtained from a mammal (e.g., a human) suspected of having cancer (e.g., brain cancer) is determined to have between 1 percent and 5 percent (e.g., from about 1 percent to about 5 percent, from about 1 percent to about 4 percent, from about 1 percent to about 3 percent, from about 1 percent to about 2 percent, from about 2 percent to about 5 percent, from about 3 percent to about 5 percent, from about 4 percent to about 5 percent, from about 2 percent to about 4 percent, from about 2 percent to about 3 percent, or from about 3 percent to about 4 percent) of the CD9⁺ EVs (e.g., CD9⁺ plasma EVs) in that sample containing a CD81 polypeptide, then that mammal can be identified as having a brain cancer (e.g., GBM).

In some cases, when a sample (e.g., a plasma sample) obtained from a mammal (e.g., a human) suspected of having cancer (e.g., brain cancer) is determined to have at least 5 percent of the CD9⁺ EVs (e.g., CD9⁺ plasma EVs) in that sample containing a CD11b polypeptide, then that mammal can be identified as having a brain cancer (e.g., GBM). For example, when a sample (e.g., a plasma sample) obtained from a mammal (e.g., a human) suspected of having cancer (e.g., brain cancer) is determined to have between 5 percent and 15 percent (e.g., from about 5 percent to about 20 percent, from about 5 percent to about 15 percent, from about 5 percent to about 10 percent, from about 10 percent to about 25 percent, from about 15 percent to about 25 percent, from about 20 percent to about 25 percent, from about 8 percent to about 22 percent, from about 10 percent to about 20 percent, from about 12 percent to about 18 percent, from about 7 percent to about 12 percent, from about 10 percent to about 15 percent, from about 15 percent to about 20 percent, or from about 18 percent to about 22 percent) of the CD9⁺ EVs (e.g., CD9⁺ plasma EVs) in that sample containing a CD11b polypeptide, then that mammal can be identified as having a brain cancer (e.g., GBM).

In some cases, when the percentage of a population of CD9⁺ EVs (e.g., CD9⁺ plasma EVs) containing a CD81 polypeptide in a sample (e.g., a blood sample) obtained from a mammal (e.g., a human) is used to identify the mammal as having brain cancer (e.g., GBM), the percentage also can be used to determine whether the brain cancer is a primary brain cancer (e.g., a primary GBM) or a metastatic brain cancer (e.g., a metastatic GBM). For example, when the percentage of a population of CD9⁺ EVs (e.g., CD9⁺ plasma EVs) containing a CD81 polypeptide in a sample (e.g., a blood sample) obtained from a mammal (e.g., a human) suspected of having brain cancer (e.g., GBM) is used to identify the mammal as having brain cancer (e.g., GBM) (e.g., is determined to have an percentage of greater than about 1 percent), the percentage also can be used to determine whether the brain cancer is a primary brain cancer (e.g., a primary GBM) or a metastatic brain cancer (e.g., a metastatic GBM). In some cases, when CD9⁺ EVs (e.g., CD9⁺ plasma EVs) containing a CD81 polypeptide in a sample (e.g., a blood sample) obtained from a mammal (e.g., a human) suspected of having brain cancer (e.g., GBM) are determined to have a percentage of from about 1 percent to about 5 percent, then that mammal can be identified as having a primary brain cancer (e.g., GBM).

In some cases, when the percentage of a population of CD9⁺ EVs (e.g., CD9⁺ plasma EVs) containing a CD81 polypeptide in a sample (e.g., a blood sample) obtained from a mammal (e.g., a human) is used to identify the mammal as having brain cancer (e.g., GBM), the percentage also can be used to determine whether the brain cancer is a pre-operative brain cancer (e.g., a pre-operative GBM) or a post-operative brain cancer (e.g., a post-operative GBM). For example, when the percentage of a population of CD9⁺ EVs (e.g., CD9⁺ plasma EVs) containing a CD81 polypeptide in a sample (e.g., a blood sample) obtained from a mammal (e.g., a human) suspected of having brain cancer (e.g., GBM) is used to identify the mammal as having brain cancer (e.g., GBM) (e.g., is determined to have an percentage of greater than about 1 percent), the percentage also can be used to determine whether the brain cancer is a pre-operative cancer (e.g., a pre-operative GBM) or a post-operative brain cancer (e.g., a post-operative GBM). In some cases, when CD9⁺ EVs (e.g., CD9⁺ plasma EVs) containing a CD81 polypeptide in a sample (e.g., a blood sample) obtained from a mammal (e.g., a human) suspected of having brain cancer (e.g., GBM) are determined to have a percentage of from about ______ percent to about ______ percent, then that mammal can be identified as having a pre-operative brain cancer (e.g., GBM).

Any appropriate method can be used to determine the percentage of EVs that contain (e.g., on a surface of the vesicle) a particular polypeptide or combination of polypeptides within a population of CD9⁺ EVs (e.g., CD9⁺ plasma EVs) in a sample (e.g., a blood sample).

For example, cytometry methods (e.g., flow cytometry such as cell sorting), spectrometry methods, and/or antibody dependent methods (e.g., enzyme-linked immunosorbent assays (ELISAs), immunoprecipitation, immunoelectrophoresis, and/or western blotting, and protein immunostaining) methods can be used to determine the percentage of EVs that contain a particular polypeptide within a population of CD9⁺ EVs (e.g., CD9⁺ plasma EVs) in a sample (e.g., a blood sample). In some cases, a percentage of EVs that contain a particular polypeptide or combination of polypeptides within a population of CD9⁺ EVs (e.g., CD9⁺ plasma EVs) in a sample (e.g., a blood sample) can be determined without enriching the EVs within the sample. In some cases, a percentage of EVs that contain a particular polypeptide or combination of polypeptides within a population of CD9⁺ EVs (e.g., CD9⁺ plasma EVs) in a sample (e.g., a blood sample) can be determined as described in Example 1.

In some cases, a population of CD9⁺ EVs (e.g., CD9⁺ plasma EVs) in a sample (e.g., a blood sample) from a mammal (e.g., a human) having brain cancer (e.g., GBM) can have an altered (e.g., increased or decreased) concentration of CD9⁺ EVs that contain (e.g., on a surface of the vesicle) one or more of a CD63 polypeptide, a CD81 polypeptide, a CD11b polypeptide, a CD41a polypeptide, a CD31 polypeptide, and a CD45 polypeptide. The term “increased concentration” as used herein with respect to a concentration of EVs (e.g., CD9⁺ EVs) that contain a particular polypeptide or combination of particular polypeptides (e.g., a CD63 polypeptide, a CD81 polypeptide, a CD11b polypeptide, a CD41a polypeptide, a CD31 polypeptide, and/or a CD45 polypeptide) refers to any concentration that is greater than the concentration of comparable EVs that contain that particular polypeptide or combination of particular polypeptides in a control sample. In some cases, an increased concentration of EVs (e.g., CD9⁺ EVs) that contain a particular polypeptide or combination of particular polypeptides (e.g., a CD63 polypeptide, a CD81 polypeptide, a CD11b polypeptide, a CD41a polypeptide, a CD31 polypeptide, and/or a CD45 polypeptide) can be at least 1 order of magnitude (e.g., 1, 2, 3, or more orders of magnitude) greater than the percentage of a comparable population of EVs that contain that particular polypeptide or combination of particular polypeptides in a control sample. The term “decreased concentration” as used herein with respect to a concentration of EVs (e.g., CD9⁺ EVs) that contain a particular polypeptide or combination of particular polypeptides (e.g., a CD63 polypeptide, a CD81 polypeptide, a CD11b polypeptide, a CD41a polypeptide, a CD31 polypeptide, and/or a CD45 polypeptide) refers to any concentration that is less than the concentration of comparable EVs that contain that particular polypeptide or combination of particular polypeptides in a control sample. In some cases, a decreased concentration of EVs (e.g., CD9⁺ EVs) that contain a particular polypeptide or combination of particular polypeptides (e.g., a CD63 polypeptide, a CD81 polypeptide, a CD11b polypeptide, a CD41a polypeptide, a CD31 polypeptide, and/or a CD45 polypeptide) can be at least 1 order of magnitude (e.g., 1, 2, 3, or more orders of magnitude) less than the percentage of a comparable population of EVs that contain that particular polypeptide or combination of particular polypeptides in a control sample. Control samples are samples obtained from normal (e.g., healthy) mammals.

In some cases, the molecular signature of a population of CD9⁺ EVs (e.g., CD9⁺ plasma EVs) in a sample (e.g., a blood sample) from a mammal (e.g., a human) having brain cancer (e.g., GBM) can have an altered (e.g., increased or decreased) concentration of the CD9⁺ EVs that contain (e.g., on a surface of the vesicle) at least one of a CD63 polypeptide, a CD81 polypeptide, a CD11b polypeptide, a CD41a polypeptide, a CD31 polypeptide, and a CD45 polypeptide.

In some cases, when a sample (e.g., a plasma sample) obtained from a mammal (e.g., a human) suspected of having cancer (e.g., brain cancer) is determined to have at least 500 events/μL of CD9⁺ EVs (e.g., CD9⁺ plasma EVs) that contain a CD11b polypeptide, then that mammal can be identified as having a brain cancer (e.g., GBM). For example, when a sample (e.g., a plasma sample) obtained from a mammal (e.g., a human) suspected of having cancer (e.g., brain cancer) is determined to have a concentration of between 500 events/μL and 7000 events/μL (e.g., from about 500 events/μL to about 7000 events/μL, from about 500 events/μL to about 6000 events/μL, from about 500 events/μL to about 5000 events/μL, from about 500 events/μL to about 4000 events/μL, from about 500 events/μL to about 3000 events/μL, from about 500 events/μL to about 2000 events/μL, from about 500 events/μL to about 1000 events/μL, from about 1000 events/μL to about 7000 events/μL, from about 2000 events/μL to about 7000 events/μL, from about 3000 events/μL to about 7000 events/μL, from about 4000 events/μL to about 7000 events/μL, from about 5000 events/μL to about 7000 events/μL, from about 6000 events/μL to about 7000 events/μL, from about 1000 events/μL to about 6000 events/μL, from about 2000 events/μL to about 5000 events/μL, from about 3000 events/μL to about 4000 events/μL, from about 1000 events/μL to about 3000 events/μL, from about 2000 events/μL to about 4000 events/μL, or from about 5000 events/μL to about 6000 events/μL) of CD9⁺ EVs (e.g., CD9⁺ plasma EVs) that contain a CD11b polypeptide, then that mammal can be identified as having a brain cancer (e.g., GBM).

Any appropriate method can be used to determine the concentration of EVs (e.g., CD9⁺ EVs) that contain (e.g., on a surface of the vesicle) a particular polypeptide or combination of polypeptides in a sample (e.g., a blood sample). For example, flow cytometry, immunofluorescence, immunostaining, and/or labeled electron microscopy methods can be used to determine the concentration of EVs (e.g., CD9⁺ EVs) that contain a particular polypeptide in a sample (e.g., a blood sample). In some cases, a concentration of EVs (e.g., CD9⁺ EVs) that contain a particular polypeptide or combination of polypeptides in a sample (e.g., a blood sample) can be determined without enriching the EVs within the sample. In some cases, a concentration of EVs (e.g., CD9⁺ EVs) that contain a particular polypeptide or combination of polypeptides in a sample (e.g., a blood sample) can be determined as described in Example 1.

In some cases, a population of CD9⁺ EVs (e.g., CD9⁺ plasma EVs) in a sample (e.g., a blood sample) from a mammal (e.g., a human) having brain cancer (e.g., GBM) can have an altered (e.g., increased or decreased) intensity (e.g., an altered MFI) of CD9⁺ EVs that contain (e.g., on a surface of the vesicle) one or more of a CD63 polypeptide, a CD81 polypeptide, a CD11b polypeptide, a CD41a polypeptide, a CD31 polypeptide, and a CD45 polypeptide. The term “increased intensity” as used herein with respect to a population of EVs (e.g., CD9⁺ EVs) that contain a particular polypeptide or combination of particular polypeptides (e.g., a CD63 polypeptide, a CD81 polypeptide, a CD11b polypeptide, a CD41a polypeptide, a CD31 polypeptide, and/or a CD45 polypeptide) refers to any intensity that is greater than the intensity of comparable EVs that contain that particular polypeptide or combination of particular polypeptides in a control sample. In some cases, an increased intensity of a population of EVs (e.g., CD9⁺ EVs) that contain a particular polypeptide or combination of particular polypeptides (e.g., a CD63 polypeptide, a CD81 polypeptide, a CD11b polypeptide, a CD41a polypeptide, a CD31 polypeptide, and/or a CD45 polypeptide) can be at least 1 order of magnitude (e.g., 1, 2, 3, or more orders of magnitude) greater than the percentage of a comparable population of EVs that contain that particular polypeptide or combination of particular polypeptides in a control sample. The term “decreased intensity” as used herein with respect to a concentration of EVs (e.g., CD9⁺ EVs) that contain a particular polypeptide or combination of particular polypeptides (e.g., a CD63 polypeptide, a CD81 polypeptide, a CD11b polypeptide, a CD41a polypeptide, a CD31 polypeptide, and/or a CD45 polypeptide) refers to any intensity that is less than the intensity of comparable EVs that contain that particular polypeptide or combination of particular polypeptides in a control sample. In some cases, a decreased intensity of a population of EVs (e.g., CD9⁺ EVs) that contain a particular polypeptide or combination of particular polypeptides (e.g., a CD63 polypeptide, a CD81 polypeptide, a CD11b polypeptide, a CD41a polypeptide, a CD31 polypeptide, and/or a CD45 polypeptide) can be at least 1 order of magnitude (e.g., 1, 2, 3, or more orders of magnitude) less than the percentage of a comparable population of EVs that contain that particular polypeptide or combination of particular polypeptides in a control sample. Control samples are samples obtained from normal (e.g., healthy) mammals.

In some cases, the molecular signature of a population of CD9⁺ EVs (e.g., CD9⁺ plasma EVs) in a sample (e.g., a blood sample) from a mammal (e.g., a human) having brain cancer (e.g., GBM) can have an altered (e.g., increased or decreased) intensity (e.g., an altered MFI) of the CD9⁺ EVs that contain (e.g., on a surface of the vesicle) at least one of a CD63 polypeptide, a CD81 polypeptide, a CD11b polypeptide, a CD41a polypeptide, a CD31 polypeptide, and a CD45 polypeptide.

In some cases, when a sample (e.g., a plasma sample) obtained from a mammal (e.g., a human) suspected of having cancer (e.g., brain cancer) is determined to have at least 1000 molecules of equivalent soluble fluorochrome (MESF) CD9⁺ EVs (e.g., CD9⁺ plasma EVs) containing at least one of a CD63 polypeptide, a CD81 polypeptide, a CD11b polypeptide, a CD41a polypeptide, a CD31 polypeptide, and a CD45 polypeptide, then that mammal can be identified as having a brain cancer (e.g., GBM). For example, when a sample (e.g., a plasma sample) obtained from a mammal (e.g., a human) suspected of having cancer (e.g., brain cancer) is determined to have an intensity of between 1000 MESF and 3750 MESF (e.g., from about 1000 MESF to about 3750 MESF, from about 1000 MESF to about 3500 MESF, from about 1000 MESF to about 3000 MESF, from about 1000 MESF to about 2500 MESF, from about 1000 MESF to about 2000 MESF, from about 1000 MESF to about 1500 MESF, from about 1500 MESF to about 3750 MESF, from about 2000 MESF to about 3750 MESF, from about 2500 MESF to about 3750 MESF, from about 3000 MESF to about 3750 MESF, from about 3500 MESF to about 3750 MESF, from about 1500 MESF to about 3500 MESF, from about 2000 MESF to about 3000 MESF, from about 1500 MESF to about 2500 MESF, from about 2000 MESF to about 3000 MESF, or from about 2500 MESF to about 3500 MESF) of CD9⁺ EVs (e.g., CD9⁺ plasma EVs) containing at least one of a CD63 polypeptide, a CD81 polypeptide, a CD11b polypeptide, a CD41a polypeptide, a CD31 polypeptide, and a CD45 polypeptide, then that mammal can be identified as having a brain cancer (e.g., GBM).

In some cases, when a sample (e.g., a plasma sample) obtained from a mammal (e.g., a human) suspected of having cancer (e.g., brain cancer) is determined to have at least 250 MESF CD11b⁺ EVs (e.g., CD11b plasma EVs) containing at least one of a CD63 polypeptide, a CD81 polypeptide, a CD11b polypeptide, a CD41a polypeptide, a CD31 polypeptide, and a CD45 polypeptide, then that mammal can be identified as having a brain cancer (e.g., GBM). For example, when a sample (e.g., a plasma sample) obtained from a mammal (e.g., a human) suspected of having cancer (e.g., brain cancer) is determined to have an intensity of between 250 MESF and 10000 MESF (e.g., from about 250 MESF to about 10000 MESF, from about 250 MESF to about 7000 MESF, from about 250 MESF to about 5000 MESF, from about 250 MESF to about 3000 MESF, from about 250 MESF to about 1000 MESF, from about 1000 MESF to about 10000 MESF, from about 3000 MESF to about 10000 MESF, from about 5000 MESF to about 10000 MESF, from about 7000 MESF to about 10000 MESF, from about 50 MESF to about 8000 MESF, from about 1000 MESF to about 7000 MESF, from about 2000 MESF to about 6000 MESF, from about 3000 MESF to about 5000 MESF, from about 1000 MESF to about 3000 MESF, from about 2000 MESF to about 4000 MESF, from about 4000 MESF to about 6000 MESF, from about 5000 MESF to about 7000 MESF, from about 6000 MESF to about 8000 MESF, or from about 7000 MESF to about 9000 MESF) of CD11b⁺ EVs (e.g., CD11b⁺ plasma EVs) containing at least one of a CD63 polypeptide, a CD81 polypeptide, a CD11b polypeptide, a CD41a polypeptide, a CD31 polypeptide, and a CD45 polypeptide, then that mammal can be identified as having a brain cancer (e.g., GBM).

In some cases, when the intensity (e.g., the MFI) of a population of CD9⁺ EVs (e.g., CD9⁺ plasma EVs) containing a CD11b polypeptide in a sample (e.g., a blood sample) obtained from a mammal (e.g., a human) is used to identify the mammal as having brain cancer (e.g., GBM), the intensity also can be used to determine whether the brain cancer is a primary brain cancer (e.g., a primary GBM) or a metastatic brain cancer (e.g., a metastatic GBM). For example, when the intensity of a population of CD9⁺ EVs (e.g., CD9⁺ plasma EVs) containing a CD11b polypeptide in a sample (e.g., a blood sample) obtained from a mammal (e.g., a human) suspected of having brain cancer (e.g., GBM) is used to identify the mammal as having brain cancer (e.g., GBM) (e.g., is determined to have an intensity of greater than about 250 MESF), the intensity also can be used to determine whether the brain cancer is a primary brain cancer (e.g., a primary GBM) or a metastatic brain cancer (e.g., a metastatic GBM). In some cases, when CD9⁺ EVs (e.g., CD9⁺ plasma EVs) containing a CD11b polypeptide in a sample (e.g., a blood sample) obtained from a mammal (e.g., a human) suspected of having brain cancer (e.g., GBM) are determined to have an intensity of from about 250 MESF to about 1250 MESF, then that mammal can be identified as having a primary brain cancer (e.g., a grade 2 glioma). In some cases, when CD9⁺ EVs (e.g., CD9⁺ plasma EVs) containing a CD11b polypeptide in a sample (e.g., a blood sample) obtained from a mammal (e.g., a human) suspected of having brain cancer (e.g., GBM) are determined to have an intensity of from about 1250 MESF to about 5000 MESF, then that mammal can be identified as having a primary brain cancer (e.g., a primary GBM). In some cases, when CD9⁺ EVs (e.g., CD9⁺ plasma EVs) containing a CD11b polypeptide in a sample (e.g., a blood sample) obtained from a mammal (e.g., a human) suspected of having brain cancer (e.g., GBM) are determined to have an intensity of from about 5000 MESF to about 10000 MESF, then that mammal can be identified as having a metastatic brain cancer (e.g., GBM).

Any appropriate method can be used to determine the intensity (e.g., MFI) of EVs (e.g., CD9⁺ EVs) that contain (e.g., on a surface of the vesicle) a particular polypeptide or combination of polypeptides in a sample (e.g., a blood sample). For example, flow cytometry and/or immunofluorescence methods can be used to determine the intensity of EVs (e.g., CD9⁺ EVs) that contain a particular polypeptide in a sample (e.g., a blood sample). In some cases, an intensity of EVs (e.g., CD9⁺ EVs) that contain a particular polypeptide or combination of polypeptides in a sample (e.g., a blood sample) can be determined without enriching the EVs within the sample. In some cases, an intensity of EVs (e.g., CD9⁺ EVs) that contain a particular polypeptide or combination of polypeptides in a sample (e.g., a blood sample) can be determined as described in Example 1.

In some cases, a size (e.g., an average longest dimension such as an average diameter) of CD9⁺ EVs (e.g., CD9⁺ plasma EVs) in a sample (e.g., a blood sample) obtained from a mammal (e.g., a human) can be used to identify the mammal as having brain cancer (e.g., GBM). For example, a sample (e.g., a blood sample) obtained from a mammal (e.g., a human) can be assessed for the size of CD9⁺ EVs (e.g., CD9⁺ plasma EVs) in the sample to determine whether the mammal has brain cancer (e.g., GBM). For example, a size (e.g., an average longest dimension such as an average diameter) of CD9⁺ EVs (e.g., CD9⁺ plasma EVs) in a sample (e.g., a blood sample) from a mammal (e.g., a human) having brain cancer (e.g., GBM) can have a different (e.g., a larger or a smaller) size of CD9⁺ EVs. The term “larger size” as used herein with respect to a size (e.g., an average longest dimension such as an average diameter) of CD9 EVs (e.g., CD9⁺ plasma EVs) refers to any size that is greater than the size of comparable EVs in a control sample. In some cases, a larger size of CD9⁺ EVs can have a size (e.g., an average longest dimension such as an average diameter) that is at least 1 order of magnitude (e.g., 1, 2, 3, or more orders of magnitude) greater than the size of comparable EVs in a control sample. The term “smaller size” as used herein with respect to a size (e.g., an average longest dimension such as an average diameter) of CD9⁺ EVs (e.g., CD9⁺ plasma EVs) refers to any size that is less than the size of comparable EVs in a control sample. In some cases, a smaller size of CD9+ EVs can have a size (e.g., an average longest dimension such as an average diameter) that is at least 1 order of magnitude (e.g., 1, 2, 3, or more orders of magnitude) less than the size of comparable EVs in a control sample. Control samples are samples obtained from normal (e.g., healthy) mammals.

In some cases, when CD9⁺ EVs that contain (e.g., on a surface of the vesicle) a CD81 polypeptide in a sample (e.g., a blood sample) obtained from a mammal (e.g., a human) suspected of having brain cancer (e.g., GBM) are determined to have a size (e.g., an average longest dimension such as an average diameter) of less than about 750 nm, then that mammal can be identified as having a brain cancer (e.g., GBM). For example, when CD9 EVs that contain (e.g., on a surface of the vesicle) a CD81 polypeptide in a sample (e.g., a blood sample) obtained from a mammal (e.g., a human) suspected of having brain cancer (e.g., GBM) are determined to have a size (e.g., an average longest dimension such as an average diameter) of from about 500 nm to about 750 nm (e.g., from about 500 nm to about 700 nm, from about 500 nm to about 650 nm, from about 500 nm to about 600 nm, from about 500 nm to about 550 nm, from about 550 nm to about 750 nm, from about 600 nm to about 750 nm, from about 650 nm to about 750 nm, from about 700 nm to about 750 nm, from about 550 nm to about 700 nm, from about 600 nm to about 650 nm, from about 550 nm to about 650 nm, or from about 600 nm to about 700 nm), then that mammal can be identified as having a brain cancer (e.g., GBM). In some cases, when CD9⁺ plasma EVs that contain (e.g., on a surface of the vesicle) a CD63 polypeptide and a CD81 polypeptide in a sample (e.g., a blood sample) obtained from a mammal (e.g., a human) suspected of having brain cancer (e.g., GBM) are determined to have a size (e.g., an average longest dimension such as an average diameter) of less than about 1400 nm, then that mammal can be identified as having a brain cancer (e.g., GBM). For example, when CD9⁺ EVs that contain (e.g., on a surface of the vesicle) a CD63 polypeptide a CD81 polypeptide in a sample (e.g., a blood sample) obtained from a mammal (e.g., a human) suspected of having brain cancer (e.g., GBM) are determined to have a size (e.g., an average longest dimension such as an average diameter) of from about 1000 nm to about 1400 nm (e.g., from about 1000 nm to about 1300 nm, from about 1000 nm to about 1200 nm, from about 1000 nm to about 1100 nm, from about 1100 nm to about 1400 nm, from about 1200 nm to about 1400 nm, from about 1300 nm to about 1400 nm, from about 1100 nm to about 1300 nm, from about 1200 nm to about 1300 nm, or from about 1100 nm to about 1300 nm), then that mammal can be identified as having a brain cancer (e.g., GBM). In some cases, when CD9⁺ plasma EVs that contain (e.g., on a surface of the vesicle) a CD45 polypeptide in a sample (e.g., a blood sample) obtained from a mammal (e.g., a human) suspected of having brain cancer (e.g., GBM) are determined to have a size (e.g., an average longest dimension such as an average diameter) of greater than about 1000 nm, then that mammal can be identified as having a brain cancer (e.g., GBM). For example, when CD9⁺ EVs that contain (e.g., on a surface of the vesicle) a CD45 polypeptide in a sample (e.g., a blood sample) obtained from a mammal (e.g., a human) suspected of having brain cancer (e.g., GBM) are determined to have a size (e.g., an average longest dimension such as an average diameter) of from about 1000 nm to about 1500 nm, then that mammal can be identified as having a brain cancer (e.g., GBM).

In some cases, when the size (e.g., an average longest dimension such as an average diameter) of CD9⁺ EVs (e.g., CD9⁻ plasma EVs) in a sample (e.g., a blood sample) obtained from a mammal (e.g., a human) are used to identify the mammal as having brain cancer (e.g., GBM), the size also can be used to determine whether the brain cancer is a primary brain cancer (e.g., a primary GBM) or a metastatic brain cancer (e.g., a metastatic GBM).

In some cases, when CD9⁺ plasma EVs that contain (e.g., on a surface of the vesicle) a CD45 polypeptide in a sample (e.g., a blood sample) obtained from a mammal (e.g., a human) suspected of having brain cancer (e.g., GBM) are used to identify the mammal as having brain cancer (e.g., GBM) (e.g., are determined to have a size (e.g., an average longest dimension such as an average diameter) of greater than about 1000 nm), the size also can be used to determine whether the brain cancer is a primary brain cancer (e.g., a primary GBM) or a metastatic brain cancer (e.g., a metastatic GBM). For example, when CD9⁺ EVs that contain (e.g., on a surface of the vesicle) a CD45 polypeptide in a sample (e.g., a blood sample) obtained from a mammal (e.g., a human) suspected of having brain cancer (e.g., GBM) are determined to have a size (e.g., an average longest dimension such as an average diameter) of from about 1000 nm to about 1500 nm, then that mammal can be identified as having a primary brain cancer (e.g., a primary GBM).

Any appropriate method can be used to determine a size (e.g., an average longest dimension such as an average diameter) of CD9⁺ EVs (e.g., CD9⁺ plasma EVs) in a sample (e.g., a blood sample) obtained from a mammal (e.g., a human) suspected of having cancer (e.g., brain cancer such as GBM). For example, size exclusion chromatography and/or light side scatter can be used to determine a size (e.g., an average longest dimension such as an average diameter) of CD9⁺ EVs (e.g., CD9⁺ plasma EVs) in a sample (e.g., a blood sample) obtained from a mammal (e.g., a human). In some cases, a size (e.g., an average longest dimension such as an average diameter) of EVs can be determined as described in Example 1.

In some cases, the characterization of CD9+ EVs (e.g., CD9+ plasma EVs) in a sample (e.g., a blood sample) obtained from a mammal (e.g., a human) suspected of having cancer (e.g., brain cancer such as GBM) can be used to identify the mammal as having brain cancer (e.g., GBM). For example, determining the presence of a population of CD9⁺ EVs having an increased percentage of CD9⁺ EVs that contain (e.g., on a surface of the vesicle) one or more (e.g., one, two, three, or more) of a CD63 polypeptide, a CD81 polypeptide, a CD11b polypeptide, a CD41a polypeptide, a CD31 polypeptide, and a CD45 polypeptide in a sample (e.g., a blood sample) obtained from a mammal (e.g., a human) can be used to identify the mammal as having brain cancer (e.g., GBM). For example, determining the presence of an increased concentration of CD9+ EVs that contain (e.g., on a surface of the vesicle) one or more (e.g., one, two, three, or more) of a CD63 polypeptide, a CD81 polypeptide, a CD11b polypeptide, a CD41a polypeptide, a CD31 polypeptide, and a CD45 polypeptide in a sample (e.g., a blood sample) obtained from a mammal (e.g., a human) can be used to identify the mammal as having brain cancer (e.g., GBM). In some cases, determining the presence of CD9⁺ EVs having a size (e.g., an average longest dimension such as an average diameter) that is different (e.g., as compared to CD9⁺ EVs from patients who do not have GBM) in a sample (e.g., a blood sample) obtained from a mammal (e.g., a human) can be used to identify the mammal as having brain cancer (e.g., GBM).

In some cases, a molecular signature and/or size of CD9⁺ plasma EVs in a sample obtained from the mammal (e.g., a human) suspected of having cancer (e.g., brain cancer) can be used to determine whether that mammal has brain cancer and, when the mammal is identified has having a brain cancer, to classify the brain cancer. For example, a sample (e.g., a blood sample) can be obtained from a mammal and assessed for a molecular signature and/or size of CD9⁺ plasma EVs in the sample as described herein to determine whether that mammal has brain cancer and, when the mammal is identified has having a brain cancer, to classify the brain cancer.

Exemplary molecular signatures that can be used to identify a mammal as having a particular condition can be as set forth below, where a plus (+) indicates an increase and a minus (−) indicates a decrease, and where a number before a plus of a minus indicates the order of magnitude of difference.

| | Molecular Signature | | | |
|---|---|---|---|---|
| | Altered | Altered | Altered | |
| | percentage | concentration | intensity | |
| Condition: | of: | of: | of: | Different EV size |
| post-operative | CD9 (1+) | | CD9 (1+) | |
| gliosis/necrosis | | | | |
| grade II | CD9 (2+), | CD9 (1+), | | CD63 (1+), CD81 |
| | CD41a (2−), | CD41a (2−), | | (1+), CD9/CD81 |
| | CD11b (1+), | CD11b+/CD45− | | (2−), |
| | CD11b+/CD45− | (3+) | | CD9/CD81/CD63 |
| | (3+) | | | (3−) |
| GBM | CD9 (3+), | CD41a (3−), | CD9 (3+), | CD63 (1+), CD81 |
| | CD41a (3−), | CD11b+/CD45− | CD11b (1+) | (1+). CD9/CD81 |
| | CD11b (1+), | (3+) | | (2−), |
| | CD9/CD81 (1+), | | | CD9/CD81/CD63 |
| | CD11b+/CD45− | | | (3−) |
| | (3+) | | | CD45 (1+) |
| metastatic | CD9 (2+), | CD9 (1+), | CD9 (2+), | CD63 (1+), CD81 |
| brain cancer | CD41a (1−), | CD41a (2−), | CD11b (1+) | (1+). CD9/CD81 |
| | CD11b (2+), | CD11b+/CD4− | | (2−), |
| | CD11b+/CD45− | (3+) | | CD9/CD81/CD63 |
| | (3+) | | | (3−) |

Exemplary molecular signatures that can be used to identify a mammal as being likely to have brain cancer (e.g., GBM) with a particular feature can be as set forth below.

| | Molecular Signature | | | |
|---|---|---|---|---|
| | Altered | Altered | Altered | Different |
| Feature: | percentage of: | concentration of: | intensity of: | EV size |
| increasing | | CD9, | | |
| tumor | | CD41a. | | |
| volume | | CD63. | | |
| | | CD9+/CD81−/ | | |
| | | CD63− | | |

Exemplary molecular signatures that can be used to identify a mammal as being likely to have a cancer with a particular outcome can be as set forth below.

| | Molecular Signature | | | |
|---|---|---|---|---|
| | Altered | Altered | Altered | Different |
| Outcome: | percentage of: | concentration of: | intensity of: | EV size |
| decreased | | CD9+. | | |
| survival | | CD9+/CD11b+ | | |
| increased | | CD9+/CD63−/ | | |
| survival | | CD81− | | |

When the characterization of CD9⁻ EVs (e.g., CD9⁺ plasma EVs) in a sample (e.g., a blood sample) obtained from a mammal (e.g., a human) is used to identify the mammal as having brain cancer (e.g., GBM) as described herein (e.g., based, at least in part, on the molecular signature and/or size of CD9⁺ plasma EVs in a sample obtained from the mammal), the determination can be confirmed using one or more additional diagnostic techniques. Examples of techniques that can be used to confirm the presence of brain cancer (e.g., GBM) can include, without limitation, neurological examinations (e.g., checking vision, hearing, balance, coordination, strength and/or reflexes), imaging tests (e.g., MRI, computerized tomography (CT), and/or positron emission tomography (PET), and laboratory tests based on tissue biopsy samples.

In some cases, a mammal (e.g., a human) identified as having brain cancer (e.g., GBM) as described herein (e.g., based, at least in part, on the molecular signature and/or size of CD9⁺ plasma EVs in a sample obtained from the mammal) can be selected for, and optionally administered or subjected to, one or more brain cancer treatments. For example, a brain cancer treatment can include any appropriate brain cancer treatment. For example, a brain cancer treatment can include one or more medical interventions. Examples of medical interventions that can be used to treat a mammal (e.g., a human) identified as having brain cancer (e.g., GBM) as described herein include, without limitation, surgery, radiation therapy (e.g., external beam radiation, proton beam therapy, intensity modulated radiotherapy, and whole brain radiation therapy), radiosurgery (e.g., stereotactic radiosurgery), and tumor treating fields (TTF; also referred to as alternating electrical fields). For example, a brain cancer treatment can include administering one or more anti-cancer drugs. In some cases, an anti-cancer drug that can be used to treat a mammal (e.g., a human) identified as having brain cancer (e.g., GBM) as described herein can be a targeted therapy. In some cases, an anti-cancer drug that can be used to treat a mammal (e.g., a human) identified as having brain cancer (e.g., GBM) as described herein can be an immunotherapeutic agent. In some cases, an anti-cancer drug that can be used to treat a mammal (e.g., a human) identified as having brain cancer (e.g., GBM) as described herein can be a chemotherapeutic agent. Examples of anti-cancer drugs that can be administered to a mammal identified as having brain cancer (e.g., GBM) as described herein can include, without limitation, temozolomide, lomustine, bevacizumab, pembrolizumab, carboplatin, etoposide, and combinations thereof.

In some cases, when a mammal (e.g., a human) is identified as having brain cancer (e.g., a GBM such as a primary GBM) as described herein (e.g., based, at least in part, on the molecular signature and/or size of CD9⁺ plasma EVs in a sample obtained from the mammal), the mammal can be selected to receive radiation therapy and to be administered temozolomide. For example, a mammal identified as having an increased percentage of CD9⁺ EVs that contain (e.g., on a surface of the vesicle) one or more (e.g., one, two, three, or more) of a CD63 polypeptide, a CD81 polypeptide, a CD11b polypeptide, a CD41a polypeptide, a CD31 polypeptide, and a CD45 polypeptide in a sample (e.g., a blood sample) obtained from the mammal can be selected to receive radiation therapy and to be administered temozolomide. For example, a mammal identified as having an increased concentration of CD9⁺ EVs that contain (e.g., on a surface of the vesicle) one or more (e.g., one, two, three, or more) of a CD63 polypeptide, a CD81 polypeptide, a CD11b polypeptide, a CD41a polypeptide, a CD31 polypeptide, and a CD45 polypeptide in a sample (e.g., a blood sample) obtained from the mammal can be selected to receive radiation therapy and to be administered temozolomide. For example, a mammal identified as having EVs having a size (e.g., an average longest dimension such as an average diameter) that is different (e.g., as compared to CD9⁺ EVs from patients who do not have GBM) in a sample (e.g., a blood sample) obtained from the mammal can be selected to receive radiation therapy and to be administered temozolomide.

In some cases, when a mammal (e.g., a human) is identified as having brain cancer (e.g., a GBM such as a metastatic GBM) as described herein (e.g., based, at least in part, on the molecular signature and/or size of CD9⁺ plasma EVs in a sample obtained from the mammal), the mammal can be selected to receive radiosurgery such as stereotactic radiosurgery (e.g., and is not administered any temozolomide). For example, a mammal identified as having an increased percentage of CD9⁺ EVs that contain (e.g., on a surface of the vesicle) one or more (e.g., one, two, three, or more) of a CD63 polypeptide, a CD81 polypeptide, a CD11b polypeptide, a CD41a polypeptide, a CD31 polypeptide, and a CD45 polypeptide in a sample (e.g., a blood sample) obtained from the mammal can be selected to receive radiosurgery such as stereotactic radiosurgery (e.g., and is not administered any temozolomide). For example, a mammal identified as having an increased concentration of CD9⁺ EVs that contain (e.g., on a surface of the vesicle) one or more (e.g., one, two, three, or more) of a CD63 polypeptide, a CD81 polypeptide, a CD11b polypeptide, a CD41a polypeptide, a CD31 polypeptide, and a CD45 polypeptide in a sample (e.g., a blood sample) obtained from the mammal can be selected to receive radiosurgery such as stereotactic radiosurgery (e.g., and is not administered any temozolomide). For example, a mammal identified as having EVs having a size (e.g., an average longest dimension such as an average diameter) that is different (e.g., as compared to CD9⁺ EVs from patients who do not have GBM) in a sample (e.g, a blood sample) obtained from the mammal can be selected to receive radiosurgery such as stereotactic radiosurgery (e.g., and is not administered any temozolomide).

In some cases, when a mammal (e.g., a human) is identified as having brain cancer (e.g., a GBM such as a primary GBM) as described herein (e.g., based, at least in part, on the molecular signature and/or size of CD9⁺ plasma EVs in a sample obtained from the mammal), the mammal can be subjected to radiation therapy and can be administered temozolomide. For example, a mammal identified as having an increased percentage of CD9⁺ EVs that contain (e.g., on a surface of the vesicle) one or more (e.g., one, two, three, or more) of a CD63 polypeptide, a CD81 polypeptide, a CD11b polypeptide, a CD41a polypeptide, a CD31 polypeptide, and a CD45 polypeptide in a sample (e.g., a blood sample) obtained from the mammal can be subjected to radiation therapy and can be administered temozolomide. For example, a mammal identified as having EVs having a size (e.g., an average longest dimension such as an average diameter) that is different (e.g., as compared to CD9⁺ EVs from patients who do not have GBM) in a sample (e.g., a blood sample) obtained from the mammal can be subjected to radiation therapy and can be administered temozolomide.

In some cases, when a mammal (e.g., a human) is identified as having brain cancer (e.g., GBM such as a metastatic GBM) as described herein (e.g., based, at least in part, on the molecular signature and/or size of CD9⁺ plasma EVs in a sample obtained from the mammal), the mammal can be subjected to radiosurgery such as stereotactic radiosurgery (e.g., and is not administered any temozolomide). For example, a mammal identified as having an increased percentage of CD9⁺ EVs that contain (e.g., on a surface of the vesicle) one or more (e.g., one, two, three, or more) of a CD63 polypeptide, a CD81 polypeptide, a CD11b polypeptide, a CD41a polypeptide, a CD31 polypeptide, and a CD45 polypeptide in a sample (e.g., a blood sample) obtained from the mammal can be subjected to radiosurgery such as stereotactic radiosurgery (e.g., and is not administered any temozolomide). For example, a mammal identified as having EVs having a size (e.g., an average longest dimension such as an average diameter) that is different (e.g., as compared to CD9⁺ EVs from patients who do not have GBM) in a sample (e.g., a blood sample) obtained from the mammal can be subjected to radiosurgery such as stereotactic radiosurgery (e.g., and is not administered any temozolomide).

When treating a mammal (e.g., a human) having brain cancer (e.g., GBM) as described herein, the treatment can be effective to reduce or eliminate the number of cancer cells present within the mammal. For example, the materials and methods described herein can be used to reduce the number of cancer cells present within a mammal having brain cancer (e.g., GBM) by, for example, 10, 20, 30, 40, 50, 60, 70, 80, 90, 95, or more percent. For example, the materials and methods described herein can be used to reduce the size (e.g., volume) of one or more tumors present within a mammal having brain cancer (e.g., GBM) by, for example, 10, 20, 30, 40, 50, 60, 70, 80, 90, 95, or more percent. In some cases, the number of cancer cells present within a mammal being treated can be monitored. Any appropriate method can be used to determine whether or not the number of cancer cells present within a mammal is reduced. For example, imaging techniques can be used to assess the number of cancer cells present within a mammal.

When treating a mammal (e.g., a human) having brain cancer (e.g., GBM) as described herein, the treatment can be effective to improve survival of the mammal. For example, the materials and methods described herein can be used to improve the survival of a mammal having cancer by, for example, 10, 20, 30, 40, 50, 60, 70, 80, 90, 95, or more percent. For example, the materials and methods described herein can be used to improve the survival of a mammal having cancer by, for example, at least 6 months (e.g., about 6 months, about 8 months, about 10 months, about 1 year, about 1.5 years, about 2 years, about 2.5 years, about 3 years, about 4 years, about 5 years, or more).

In some cases, the characterization of CD9⁺ EVs (e.g., CD9⁺ plasma EVs) in a sample (e.g., a blood sample) obtained from a mammal (e.g., a human) can be used to identify the mammal as not having brain cancer (e.g., GBM). For example, the absence of an increased percentage of CD9⁺ EVs that contain (e.g., on a surface of the vesicle) a CD63 polypeptide, a CD81 polypeptide, a CD11b polypeptide, a CD41a polypeptide, a CD31 polypeptide, and a CD45 polypeptide in a sample (e.g., a blood sample) obtained from a mammal (e.g., a human) can be used to identify the mammal as not having brain cancer (e.g., GBM). For example, the presence of EVs having a size (e.g., an average longest dimension such as an average diameter) that is not different (e.g., as compared to CD9⁺ EVs from patients who do not have GBM) in a sample (e.g, a blood sample) obtained from a mammal (e.g., a human) can be used to identify the mammal as not having brain cancer (e.g., GBM).

In some cases, a mammal (e.g., a human) identified as not having brain cancer (e.g., GBM) as described herein (e.g., based, at least in part, on the molecular signature and/or size of CD9⁺ plasma EVs in a sample obtained from the mammal) is not selected for or administered any cancer treatment.

In some cases, a mammal (e.g., a human) identified as not having brain cancer (e.g., GBM) as described herein (e.g., based, at least in part, on the molecular signature and/or size of CD9⁺ plasma EVs in a sample obtained from the mammal) can be selected for increased monitoring for one or more other conditions (e.g., brain conditions that can have non-specific findings on MRI such as brain abscesses, tumefactive multiple sclerosis, and subacute infarction).

In some cases, a mammal (e.g., a human) identified as not having brain cancer (e.g., GBM) as described herein (e.g., based, at least in part, on the molecular signature and/or size of CD9⁺ plasma EVs in a sample obtained from the mammal) can be selected for further testing (e.g., further diagnostic testing) for other conditions (e.g., brain conditions that can have non-specific findings on MRI such as brain abscesses, tumefactive multiple sclerosis, and subacute infarction).

The invention will be further described in the following examples, which do not limit the scope of the invention described in the claims.

### EXAMPLES

### Example 1: A Distinct Plasma EV Phenotype in GBM Patients

Flow cytometry was used to demonstrate differences in bulk plasma EV phenotype between samples from GBM patients and normal donors (ND) that could serve as a liquid biopsy assay. Plasma EVs were stained for EV-associated tetraspanins (CD9/CD63/CD81), markers indicating cell of origin (CD11b/CD31/CD41a/CD45), and actin/phalloidin (to exclude cell debris). EVs were analyzed using spectral flow cytometry. Multiparametric analysis using t-distributed stochastic neighbor embedding (t-SNE) and self-organizing maps on flow cytometry data (FlowSOM) was performed comparing GBM and normal donor plasma EVs. Size exclusion chromatography plus modified flow cytometer threshold settings enriched plasma EVs while minimizing background noise. GBM patients had increased CD9⁺, CD81⁺, and myeloid-derived (CD11b⁺) EVs. Multiparametric analysis demonstrated distinct surface marker expression profiles in EVs in plasma from GBM patients compared to EVs in plasma from NDs. Fifteen plasma EV sub-populations differing in size and surface marker expression were identified, three enriched in plasma from GBM patients and three enriched in plasma in normal donors. Multiparametric analysis demonstrates that GBM patients have a distinct plasma EV phenotype compared to ND.

### Materials and Methods

### GBM Patient and Normal Donor Blood and Plasma

GBM (isocitrate dehydrogenase (IDH) wildtype) patient plasma samples and normal donor plasma samples were obtained. Blood samples were collected in ethylenediaminetetraacetic acid tubes and centrifuged twice (2000 g×10 minutes) to remove cells and harvest plasma. Plasma samples were further centrifuged (1500 g×10 minutes and 15000 g×10 minutes) to remove any additional cell debris and platelets. These samples were stored in sterile cryogenic vials (Corning Incorporated No. 430488) at −80 °C for the following experiments.

### Spectral Flow Cytometry Calibration

A 5-laser Cytek® Aurora Flow Cytometer equipped with SPECTROFLO® software (Cytek Biosciences Inc, Fremont, CA) was used. ApogeeMix beads (Cat #1527, Apogee Flow Systems, United Kingdom) were used for calibration and optimizing acquisition settings. Serial dilution was performed to demonstrate detection of single particles. Comparison was made between side scatter (SSC) and various fluorescent channels to optimize thresholds for nanoparticle acquisition.

### Isolating CD14⁺ Monocyte-Derived EVs

Peripheral blood mononuclear cells (PBMC) were obtained from discarded anonymized healthy donor leukoreduction chambers via Ficoll gradient centrifugation (800 g×15 minutes). CD14⁺ monocytes were isolated from PBMC with CD14⁺ magnetic beads (Miltenyi Biotec, San Diego, CA) per manufacturer instructions. CD14⁺ cells (1×10⁶ per well) were seeded in 6 well plates (Corning Incorporated; Corning, NY) in RPMI (Corning, Mediatech Inc., Manassas, VA) media with 10% fetal bovine serum (FBS) (Atlanta Biologicals, Flowery Branch, CA) and 1% penicillin/streptomycin (PEN) Solution (Sigma-Aldrich, St. Louis, MO) and incubated at 37 °C with 5% CO₂. After 24 hours, media was replaced with serum-free RMPI, incubated for an additional 72 hours, collected, and centrifuged (1200 RPM×10 minutes twice) to remove cells and cell debris. The supernatant was ultracentrifuged at 24000 RPM for 16 hours at 4 °C (Optima LE-80K Ultracentrifuge, Beckman Coulter, Indianapolis, IN) using a swinging bucket (SW 55 Ti, Beckman Coulter, Indianapolis, IN). Supernatant except for the last 300 μL was discarded. Nanoparticle tracking analysis (Nanosight) was performed to determine the concentration of enriched EV samples.

### Antibodies

Detailed information for all antibodies utilized in flow cytometry and western blot experiments is listed in Table 1.

TABLE 1
Antibodies for Flow cytometry analysis

| | | | | Catalogue | Volume/ | | |
|---|---|---|---|---|---|---|---|
| Reagent | Fluorochrome | Vendor | Clone | # | test (μL) | | Brightness* |
| CD 11b | PE | eBioscience | ICRF44 | 12-0118-73 | 1 | | 5 |
| CD 11b | Pacific Blue | BioLegend | M1/70 | 101224 | 1 | | 1 |
| CD 11b | PE-Cy7 | BioLegend | M1/70 | 101216 | 1 | (0.2 μg) | 4 |
| CD 11b | BV785 | BioLegend | ICRF44 | 301346 | 1 | | 3 |
| CD31 | APC-Cy7 | BD | WM59 | 563653 | 1 | (0.1 μg) | 2 |
| | | Biosciences | | | | | |
| CD31 | APC | BioLegend | WM59 | 303116 | 1 | | 5 |
| CD45 | BV650 | BioLegend | HI30 | 304044 | 1 | | 4 |
| CD45 | BV510 | BioLegend | HI30 | 304036 | 1 | (0.1 μg) | 4 |
| Phalloidin- | iFluor 405 | Abcam | — | ab176752 | 0.1 | (0.1 μg) | ? |
| iFluor 405 | | | | | | | |
| CD81 | Alexa Fluor 647 | RD Systems | 454720 | FAB4615R | 1 | (0.2 μg) | 5 |
| CD41a | PE | BioLegend | HIP8 | 303706 | 1 | (0.0125 μg) | 5 |
| CD9 | Alexa Fluor 488 | RD Systems | 209306 | FAB1880G | 1 | (0.2 μg) | 3 |
| CD63 | PE-Cy5 | Abcam | MEM-259 | ab234251 | 1 | (0.055 μg) | 4 |

| | | | | |
|---|---|---|---|---|
| FC Block | BD | 3216, | 564219 | 1 |

| | |
|---|---|
| Biosciences | 564219 |

Reagents included in the final panel are in bold face type.
*Brightness intensity data is from fluorochromes brightness chart. (See, for example, bbrightnessu.edu/flow-cytometry/files/2013/06/Fluorochromes-Brightness-Chart.pdf)

### Developing an EV Flow Cytometry Characterization Panel

Initial assay controls were performed using CD14⁺ monocyte-derived EVs. Phosphate-buffered saline (PBS; JX, GE Healthcare Life Sciences Hyclone Laboratories, Logan, UT) was passed through a 100 nm filter (Anotop™ 10, GE healthcare UK limited, UK) and degassed. CD14⁺ monocyte-derived EVs were stained by diluting 20 μL EVs in 80 μL PBS and incubating with 1 μL of anti-CD9 and/or CD11b antibody (1:100 dilution). Assay controls included buffer only (PBS), buffer with reagent (PBS+antibody), single color staining (CD9 or CD11b), and detergent-treated EV staining control (02 SDS) per International Society for Extracellular Vesicles (ISEV) guidelines (see, e.g., Welsh et al., J. Extracell. Vesicles, 9:1713526 (2020)). Serial dilution of CD9-stained EV samples was performed. Subsequent assay controls were performed with plasma samples. An antibody mix including anti-CD9, CD11b, CD31, CD41a, CD45, CD63, and CD81 antibodies, phalloidin, and Fc blocking buffer (see Table 1 for relative volumes) was spun (21000 g×10 minutes) to remove antibody aggregates. 50 μL plasma samples were added to 50 μL antibody mix, then incubated in the dark at room temperature for 60 minutes. CD9 titration for plasma samples was performed by staining 50 μL plasma sample with different amounts of CD9 antibodies. PBS only, PBS+all antibodies, single color staining, all color staining, and fluorescence minus one controls were performed for all markers. Strongly fluorescent fluorochromes compatible with the overall panel were chosen. Comparison was made between stained plasma samples and PBS+antibody controls to identify antibodies with the highest signal to noise ratio.

### Separating and Concentrating Stained EV Samples

Differential ultracentrifugation, (DU) density gradient ultracentrifugation (DGU), size exclusion chromatography (SEC), and ultrafiltration (UF) were compared for separating and concentrating stained EVs while removing unstained antibodies. For DU, stained EVs (100 μL) in 4 mL PBS were ultracentrifuged (100000 g×90 minutes; Beckman Coulter Optima LE-80K Ultracentrifuge, Rotor SW55Ti). All supernatant was discarded except for the last 200 μL. For DGU, stained EVs (100 μL) in 3 mL PBS were layered on top of 1 mL of 0.971 M sucrose in an ultra-clear ultracentrifugation tube (13*51 mm, Beckman Coulter, Indianapolis, IN), then ultracentrifuged (190000 g×2 hours). The EV pellet was resuspended in PBS (200 μL). For UF, stained EVs (100 μL) in 14 mL of PBS and concentrated to 500 μL with concentration filters (Amicon® Ultra-15, 10 kDa MWCO, Sigma-Aldrich, St. Louis, MO). For SEC, stained EVs (100 μL) were run through qEV single/70 nm columns (IZON Science, Portland, OR). EV fractions were collected per manufacturer's instructions. Purified samples were analyzed on a Cytek flow cytometer with stopping criteria set as a 20 μL sample collection and a SSC-500 threshold. For each method, PBS+antibody was used as a process control. Finally, SEC to separate/concentrate stained EVs was further tested in plasma samples.

### Nanoparticle Tracking Analysis (NTA)

Particle concentration in cell culture-derived EVs and stained plasma EVs was analyzed using NanoSight (Malverin Panalytical, NanoSight NS300, Westborough MA). Plasma samples were diluted 1:50 and cell culture-derived EVs were diluted 1:100 in filtered PBS prior to NTA. Triplicate measurements (collection time 30 seconds/each; camera level 15) were analyzed using NTA software (Malvern Panalytical, Westborough MA, Version number MAN0545-01-EN-00) with the threshold of 5.

### Western Blots

Samples were prepared with RIPA buffer (50 mM Tris (pH 7.4), 1% Triton X100, 0.25% Sodium deoxycholate, 150 mM NaCl, 1 mM EDTA (pH 8) and 10 mM NaF). Proteins were separated by electrophoresis on SDS-PAGE, followed by membrane transfer and probing with primary antibodies (CD9, CD81, and CD63). Secondary antibodies were horseradish peroxidase-conjugated goat anti-rabbit or goat anti-mouse. Membranes were visualized by enhanced chemiluminescence. Antibodies used are indicated in Table 2.

TABLE 2
Antibodies for western blot analysis

| | | Catalogue |
|---|---|---|
| Reagent | Vendor | Number |
| HSP90 Rabbit Ab | Cell Signaling | 4874S |
| CD9 Rabbit mAb | Cell Signaling | 13174S |
| CD81 Rabbit mAb | Cell Signaling | 56039S |
| Anti-CD63 Antibody | Santa Cruz | sc-5275 |
| (MX-49.129.5) | | |
| CD11b Rabbit mAb | Cell Signaling | 49420S |
| Goat Anti-Rabbit IgG | Jackson | 111-035-003 |
| | ImmunoResearch | |

### Flow Cytometry Data Acquisition and Analysis

After performing daily quality controls, the flow cytometer was cleaned with 10% bleach followed by molecular grade water and filtered PBS at low speed (10 minutes each). Stained, purified plasma EV samples (20 μL) diluted in 1 mL filtered PBS were analyzed. EV data was collected on the Cytek Aurora flow cytometer with an SSC-500 threshold and a 100 μL stopping criteria for sample collection. Subsequently, thresholds were changed to the fluorescent intensity peak channels for each fluorophore (V7/B2/YG1/YG5/YG9/R2/R7-600). At least 8×10⁴ CD9⁺ events were collected. Events were manually gated to exclude background noise and only events in the EV size range (gated as “EV region”) were included. Flow data was analyzed in FlowJo™ Software for Windows Version 10.8.1 (FlowJo LLC; Ashland, OR). Further gating for CD9⁺/phalloidin⁻ events was performed. Finally, self-organizing map clustering via the FlowSOM algorithm were performed and dimensionality reduction using t-distributed stochastic neighbor embedding (tSNE) on a combined data set. This combined data set contained 8×10⁴ randomly sampled phalloidin-negative events from each replicate (n=10 each for GBM and normal donors). tSNE and subsequent FlowSOM analysis incorporated SSC-A, CD9, CD31, CD11b, CD45, CD41a, CD63, and CD81. Metaclustering was further applied to these samples and an additional 10 GBM and 10 ND replicates (n=20 total replicates/condition) to determine the frequency of each cluster.

### Statistical Analysis

Student t-test (parametric) and Mann-Whitney test (non-parametric) were used to compare two groups. All data were analyzed and plotted in GraphPad Prism version 9.2.0 for Windows (GraphPad Software, San Diego, CA).

### Results

### Optimizing Spectralflow Cytometry for Nanoparticle Analysis

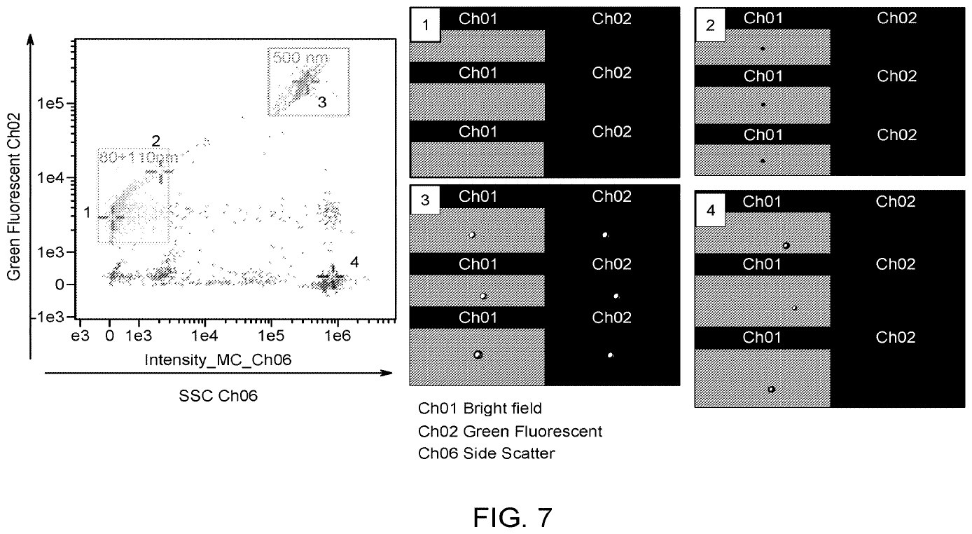

ApogeeMix calibration beads including data acquisition settings for gain were FSC 20, SSC 1000, and SSC-B 1000. Similar analysis using an ImageStream®X MkII Imaging Flow Cytometer (Amnis Corporation; Seattle, WA) demonstrated individual particles but had poor resolution for non-fluorescent beads (FIG. 7).

### Developing an EV Staining Protocol

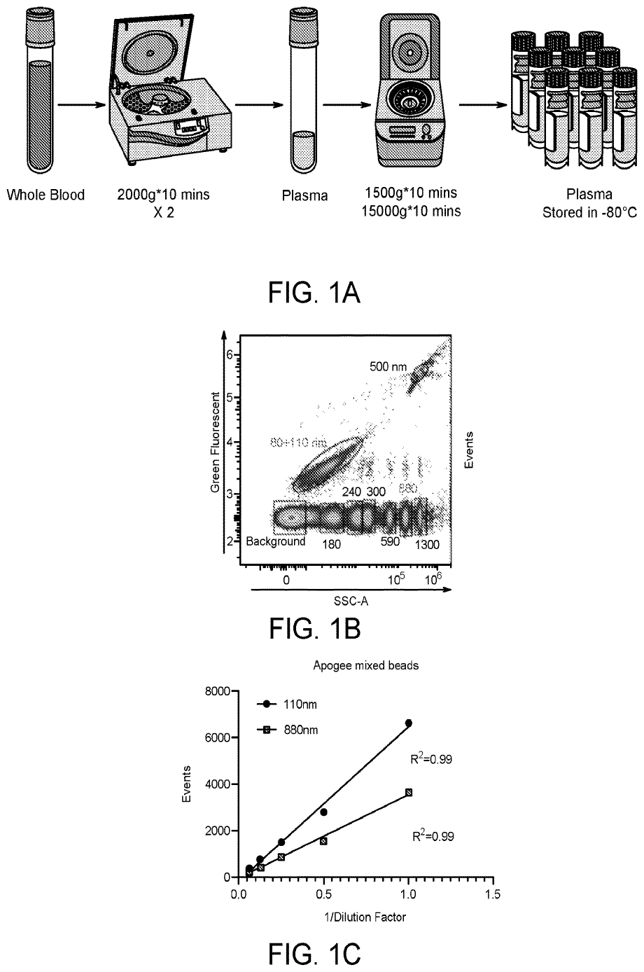

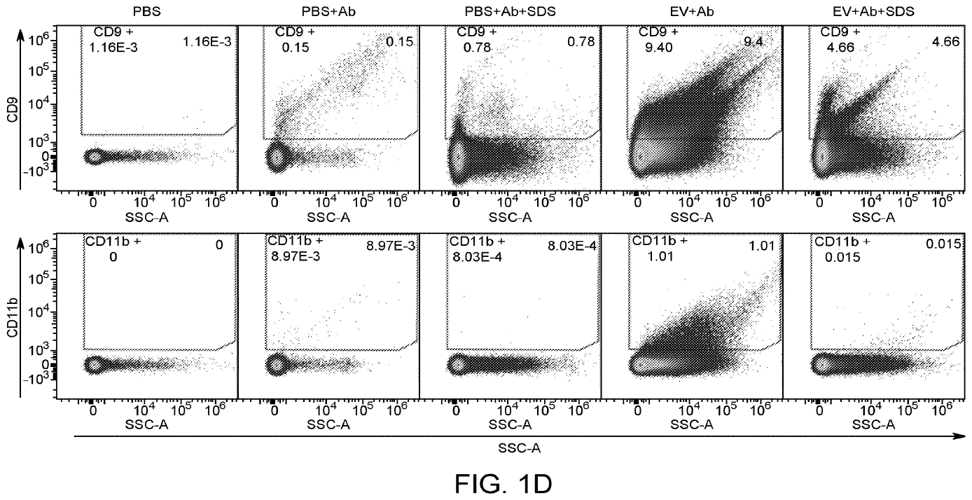

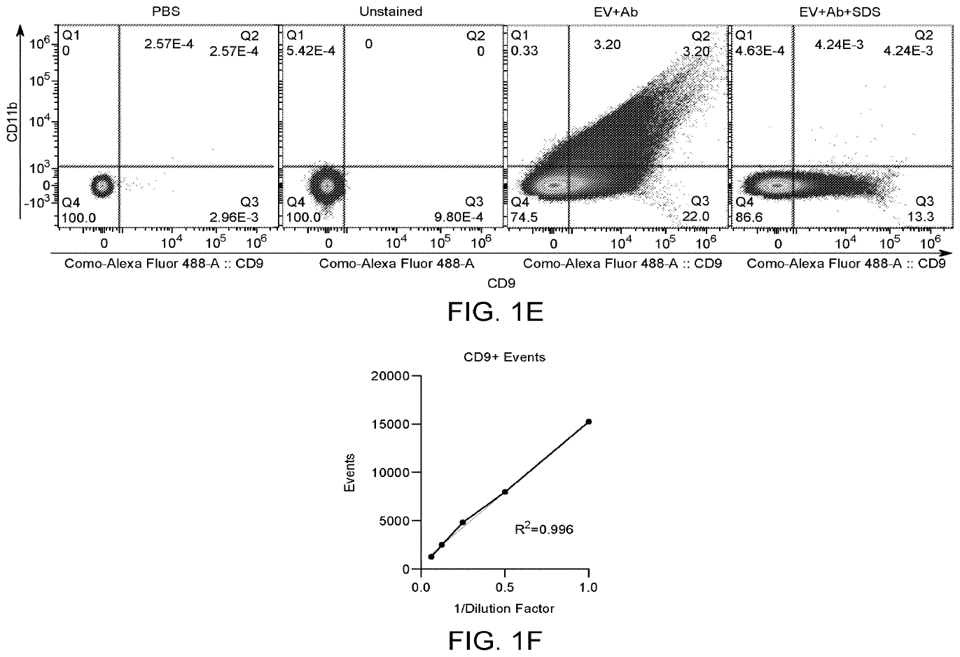

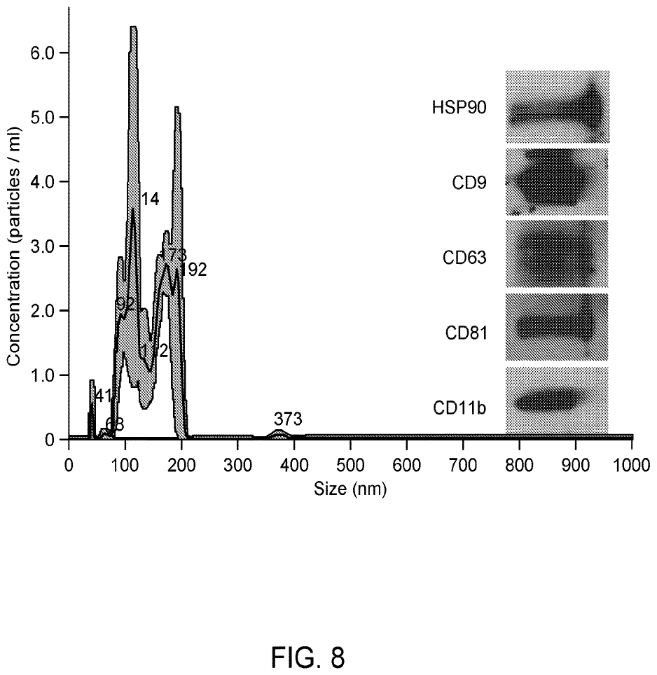

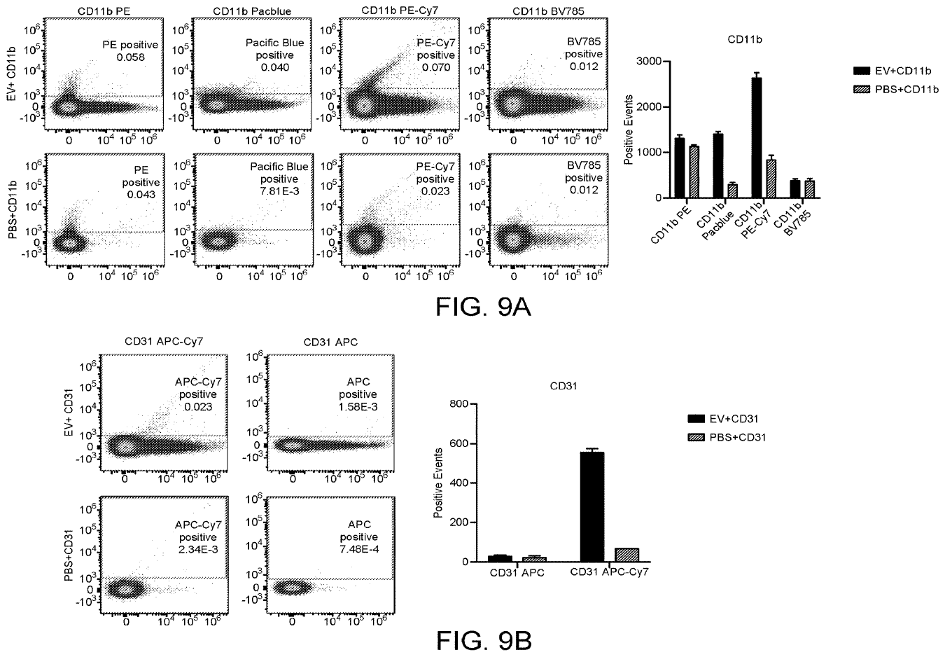

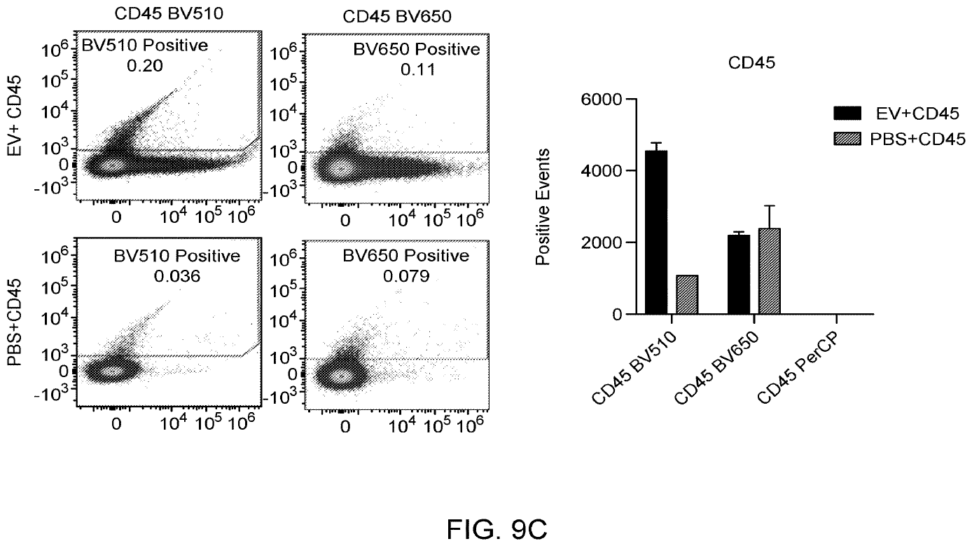

Since plasma contains heterogeneous particles in which the EV concentration remains largely unknown, an in vitro EV staining protocol was developed with monocyte-derived EVs and single color staining for CD9 and CD11b (FIG. 1D). Nanoparticle tracking confirmed the presence of EVs and while western blot demonstrated EV-associated tetraspanin (CD9, CD63, and CD81), CD11 b, and HSP-90 expression (FIG. 8). There was a small false positive signal in PBS+Ab controls that was not eliminated by 0.2% SDS. However, false positive frequency was much lower than true positive events (CD9: 0.15% vs 9.4%; CD11b: 0.009% vs 1.01%). Exposure to detergent (0.2% SDS) eliminated most of positive events, indicating their vesicular nature. Dual color CD9 and CD11 staining showed the majority of CD11b positive events were also positive for CD9 though there were also many CD11 b⁻/CD9⁺ events (FIG. 1E). There was a linear relationship between dilution factor and CD9⁺ events suggesting detection of single events (R2=0.99) (FIG. 1F). Finally, antibodies with different fluorochromes were compared for a given surface marker to maximize signal to noise ratio (FIGS. 9A-9C).

### Indeterminate EV Identification Using Membrane Dyes

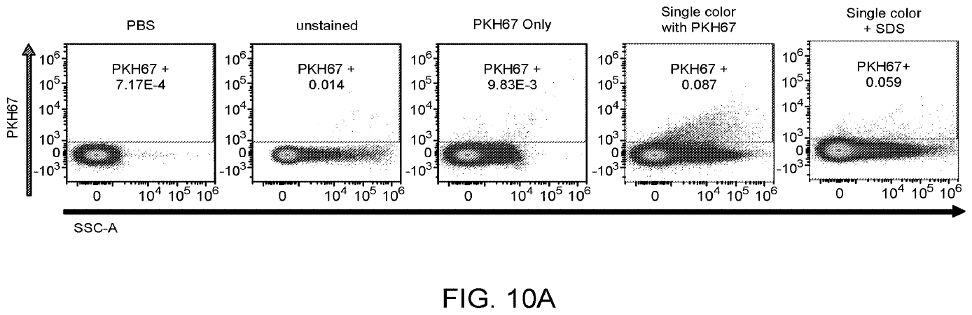

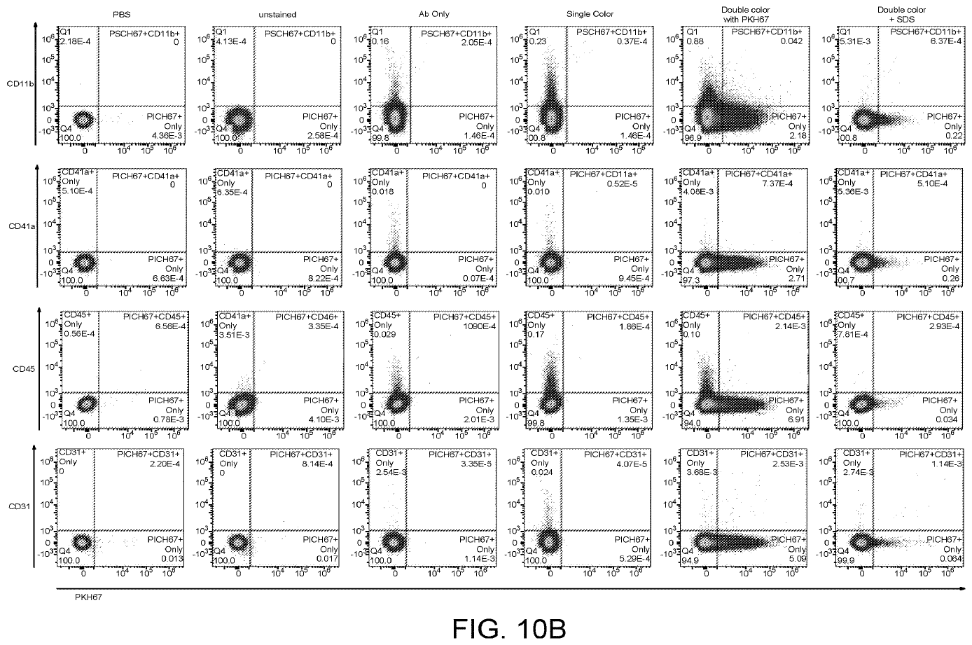

Initially, the membrane fluorescence dye PKH67 (Sigma-Aldrich, St. Louis, MO) was tested as a universal EV marker (FIGS. 10A-10B). This showed low false positive events in reagent-only controls and increased positive events when EVs were added that were substantially reduced by 0.2% SDS treatment, suggesting a vesicular nature. However, two-color staining with PKH67 and various surface markers (CD11b, CD41a, CD45, and CD31) was less conclusive. There was a significant increase in corresponding positive events for each surface marker in single-color staining compared to antibody-alone controls but two-color staining with PKH67 dye showed few double positive events. Treating double-stained EVs with SDS eliminated most of the surface marker-positive and PKH67-positive events. This could indicate PKH67 stains both EV particles and non-EV particles in the plasma. Thus, staining with PKH67 was discontinued as a universal plasma EV labeling strategy in favor of anti-tetraspanin antibodies.

### Size Exclusion Chromatography is Most Efficient for Separating and Concentrating Stained Plasma EVs

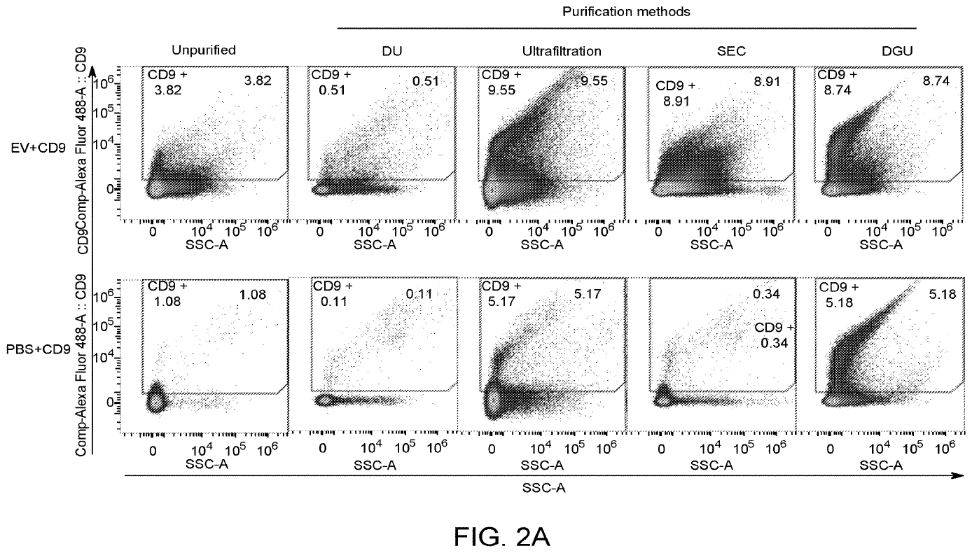

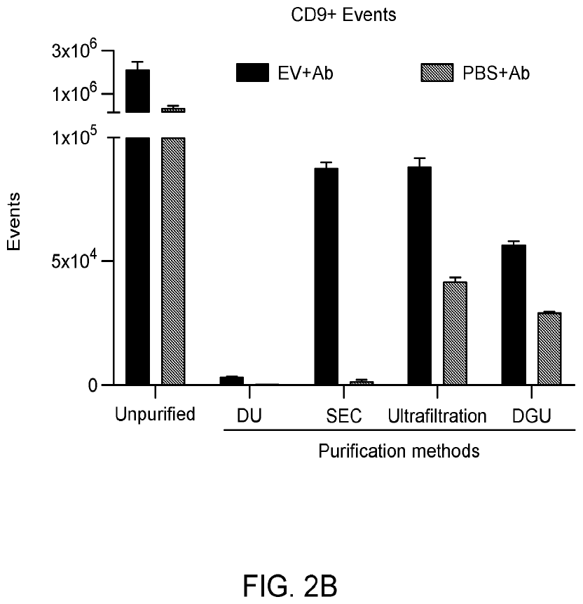

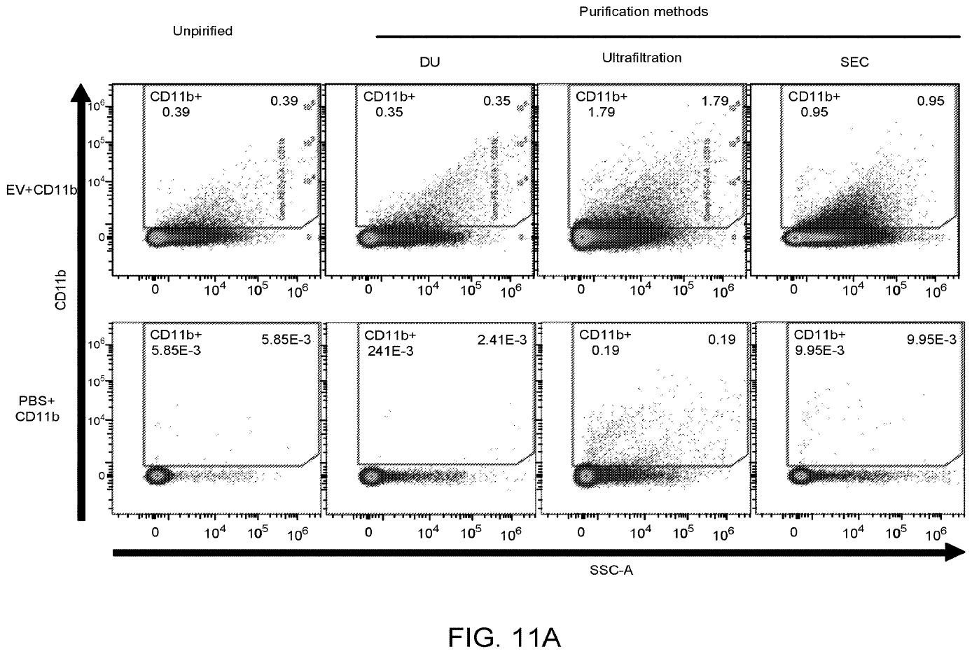

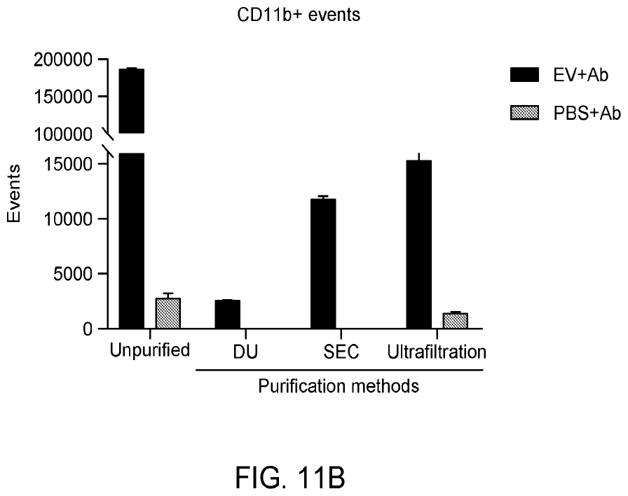

Four standard EV separation and concentration methods were compared: differential ultracentrifugation, ultrafiltration, size-exclusion chromatography, density gradient ultracentrifugation (FIG. 2). PBS+CD9 antibody controls were used for each method. Differential ultracentrifugation removed most false positive events (1.08% vs 0.11%) but also reduced CD9⁺ EVs (3.82% vs 0.51%). Ultrafiltration and DGU purification enriched both CD9⁺ EVs (9.55% vs 3.82%, 8.74% vs 3.82%, respectively) and false-positive events (5.17% vs 1.08%, 5.18% vs 1.08%, respectively). SEC produced the highest signal-to-noise ratio (8.91% vs 0.34%). Furthermore, while all four methods lowered the total CD9⁺ EV yield, this reduction was smallest for size exclusion chromatography (along with ultrafiltration). Repeated experiments using EVs stained for a different surface marker (CD11b) also showed that SEC-purified samples had the highest signal-to-noise ratio (0.95% vs 0.009%) with relatively preserved yield (FIGS. 11A-11B). The SEC protocol was chosen as a preferred purification method for plasma sample staining.

### Developing a Staining Panel for Plasma EV Flow Cytometry Analysis

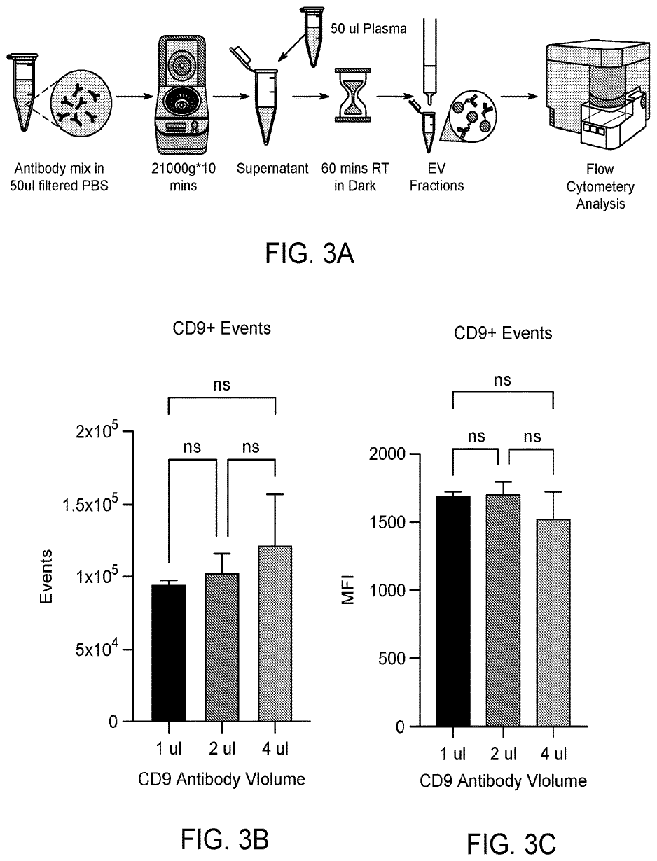

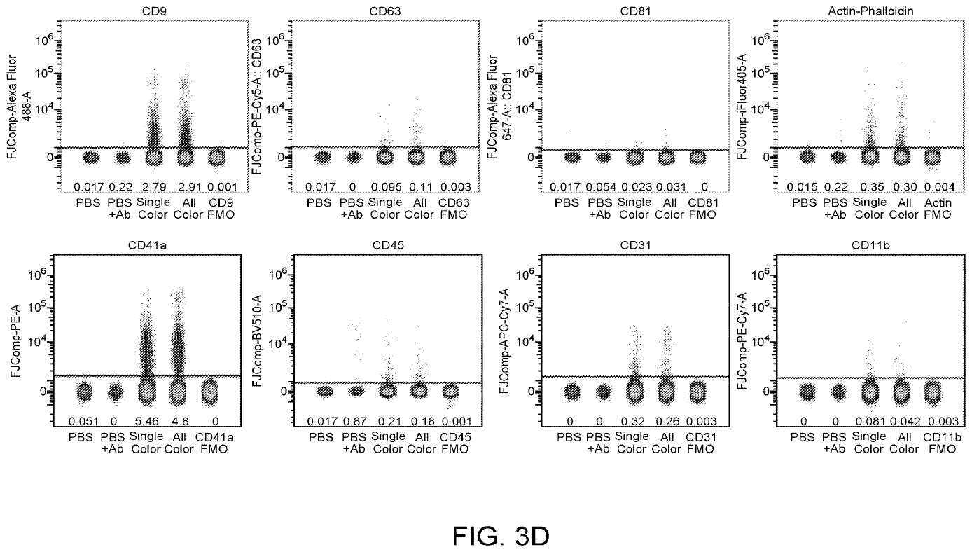

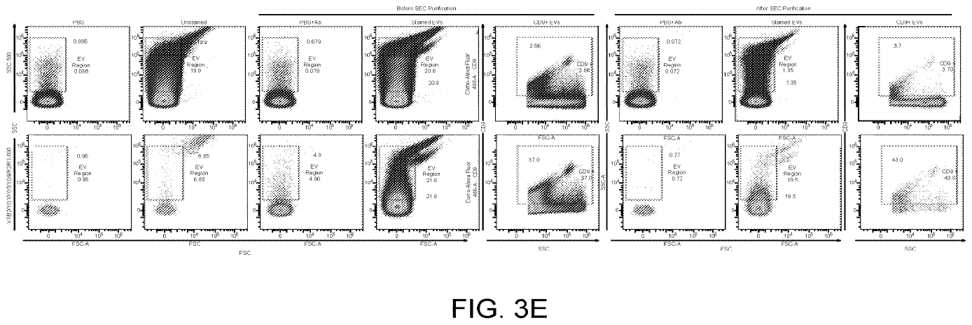

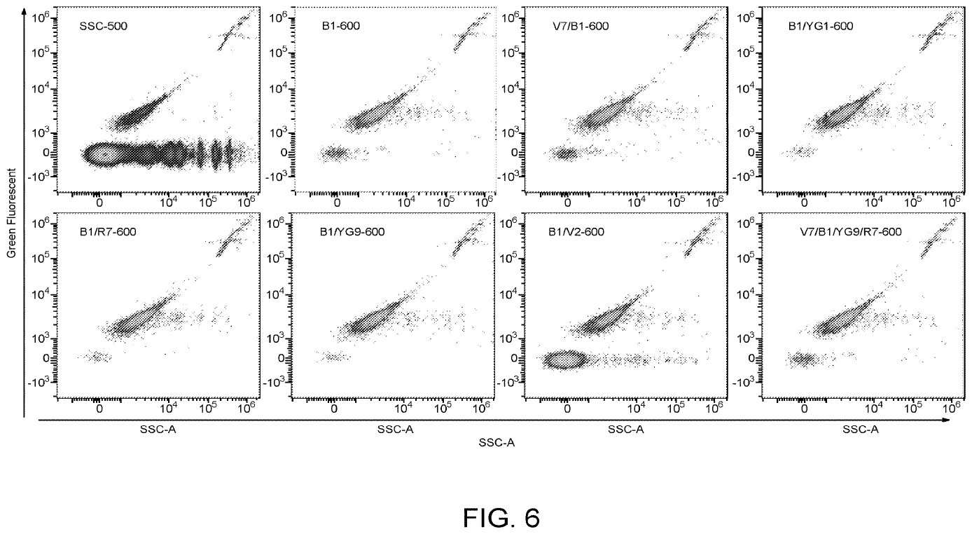

An exemplary protocol for plasma EV staining followed by SEC for EV separation and concentration is shown in FIG. 3A. In addition to EV-associated tetraspanins (CD9, CD83, and CD81), EVs originating from myeloid cells (CD11b⁺), leukocytes (CD45⁺), platelets (CD31⁺/CD41a⁺), and endothelial cells (CD31⁺/CD41a⁻) were identified and excluded particles staining for actin (phalloidin⁻) representing cellular debris. Antibody titration was performed to demonstrate the lowest amount of antibody that reliably produced similar event rates and mean fluorescence intensity (MFI) (FIGS. 3B and 3C). Assay controls for each marker included PBS alone, PBS+antibody, EVs+single antibody, EVs+all antibodies, and EVs+all antibodies except the stain of interest (fluorescence minus one). CD9 and CD41a were the most abundantly expressed surface markers in plasma EVs identified by the panel (FIG. 3D). However, most EV-sized particles detected in plasma with the initial SSC-500 acquisition threshold did not express any surface makers and likely represented contaminating non-EV particles. To minimize this, different acquisition thresholds were assessed. An SSC-500 acquisition threshold was initially chosen as the assays with microbead showed this enabled detection of both the fluorescent and nonfluorescent beads while thresholds based on fluorescent spectra only detected fluorescent beads (FIG. 6). However, this was less of an issue with fluorescent-labeled, SEC-purified plasma EVs where changing from an SSC-500 acquisition threshold to a spectral fluorescence intensity threshold based on all the remaining fluorochromes (V7/B2/YG1/YG5/YG9/R2/R7-600) minimized the detection of both background noise and non-EV particles (FIG. 3E).

### Analyzing Plasma EVs in GBM Patients and Normal Donors

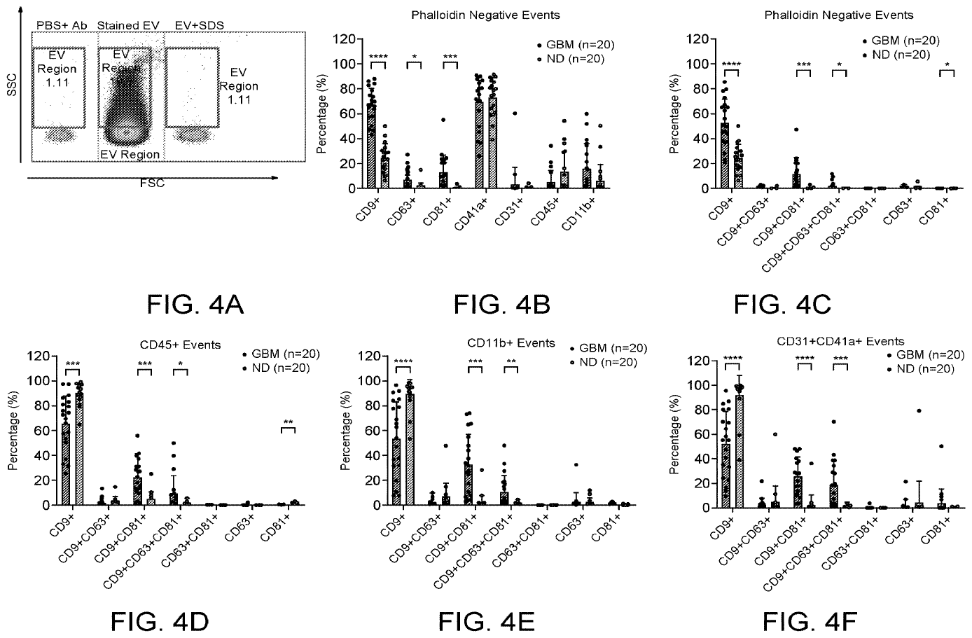

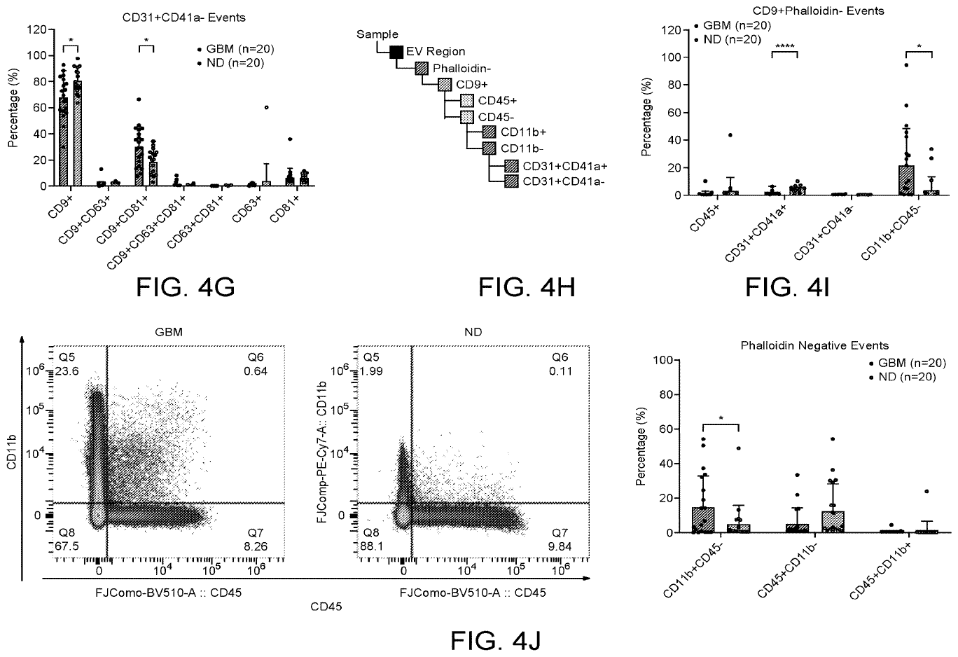

Phalloidin-negative particles in the EV size range were gated (FIG. 4A), followed by gating for other surface markers. CD41a⁺ and CD9⁺ EVs were the most common in GBM patients and normal donors (FIG. 4B). There were no significant differences in CD41a⁺ EVs (mean 70.00% vs 73.04%, p=0.59), CD31⁺ EVs (mean 3.72% vs 0.59%, p=0.51), or CD45⁺ EVs (mean 5.38% vs 13.67%, p=0.21) in GBM patients versus normal donors. GBM patients trended toward higher CD11b⁺ EVs (mean 16.15% vs 6.37%, p=0.21) and had significant increases in CD9⁺ EVs (mean 67.08% vs 24.83%, p<0.0001), CD63⁺ EVs (mean 7.22% vs 1.99%, p=0.04), and CD81 EVs (mean 13.5% vs 0.37%, p=0.0007) (FIG. 4B). Among EV-associated tetraspanins, most plasma EVs only expressed CD9 (FIG. 4C). GBM patients had increased CD9⁺ (mean 52.7% vs 24.33%, p<0.00001), CD9′CD81⁻ (mean 11.25% vs 0.24% p<0.001), and CD9⁺CD63⁺CD81⁺ EVs (mean 2.48% vs 0.05%, p=0.02). Most CD45′, CD11b⁺, CD31′CD41a⁺, and CD31′CD41a⁻ EVs in normal donors were CD9⁺CD63⁻CD81⁻, while in GBM patients they could be CD9⁺, CD9⁻CD81⁺, or CD9⁺CD63⁺CD81⁺ (FIGS. 4D-4G). Further gating of CD9′/phalloidii EVs was performed to deduce their cells of origin (FIG. 4H). GBM patients had increased CD9⁺CD11b⁺CD45⁻ EVs (putative myeloid-derived EVs) compared to normal donors (mean 21.69% vs 3.9%, p=0.02) (FIG. 4I), while normal donors had increased CD9⁺CD31⁺CD41a⁺ EVs (platelet-derived EVs, mean 1.05% vs 4.43%, p<0.0001). Interestingly, further gating of phalloidin-negative events based on CD45 and CD11b expression showed most positive events were single positives for either CD45 or CD11b (FIG. 4J). Unlike CD11b⁺ myeloid cells, very few double positive CD45⁺/CD11b⁺ EVs were seen. However, GBM patients again had significantly higher percentage of CD11b⁺CD45⁻ EVs compared to normal donors (mean 14.84% vs 4.72%, p=0.037).

### Differentially Expressed EV Subpopulations in GBM Patients and Normal Donors

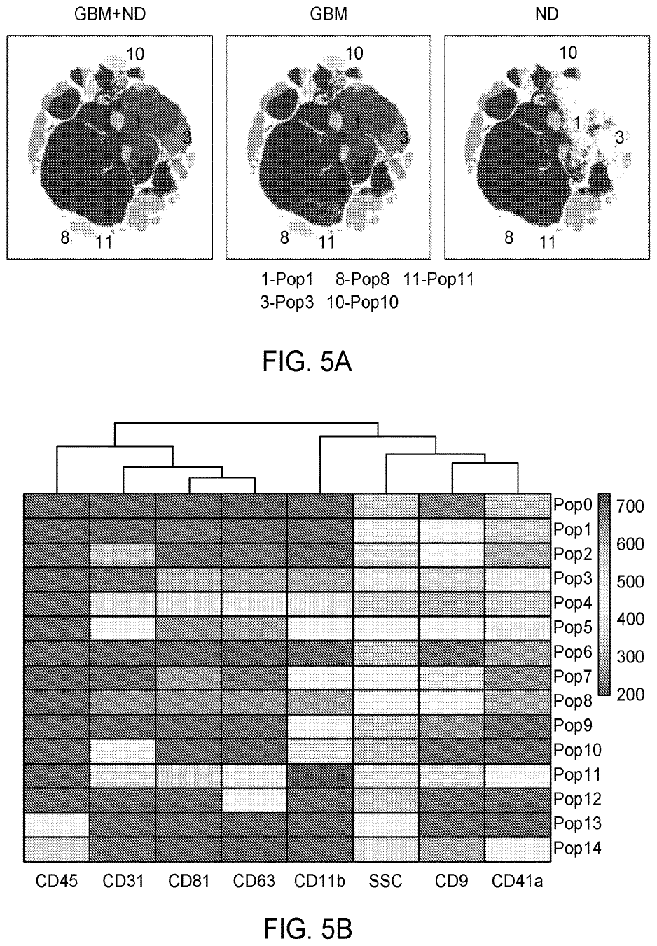

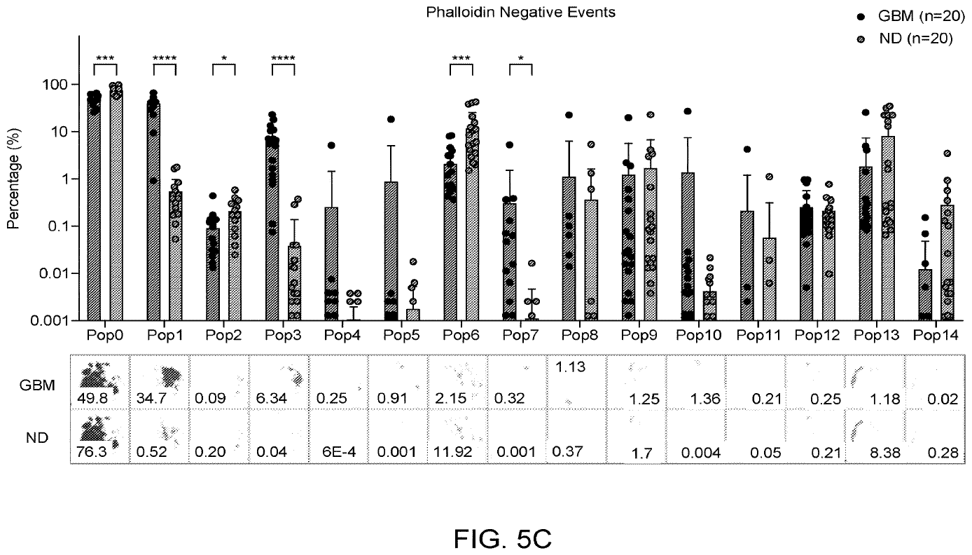

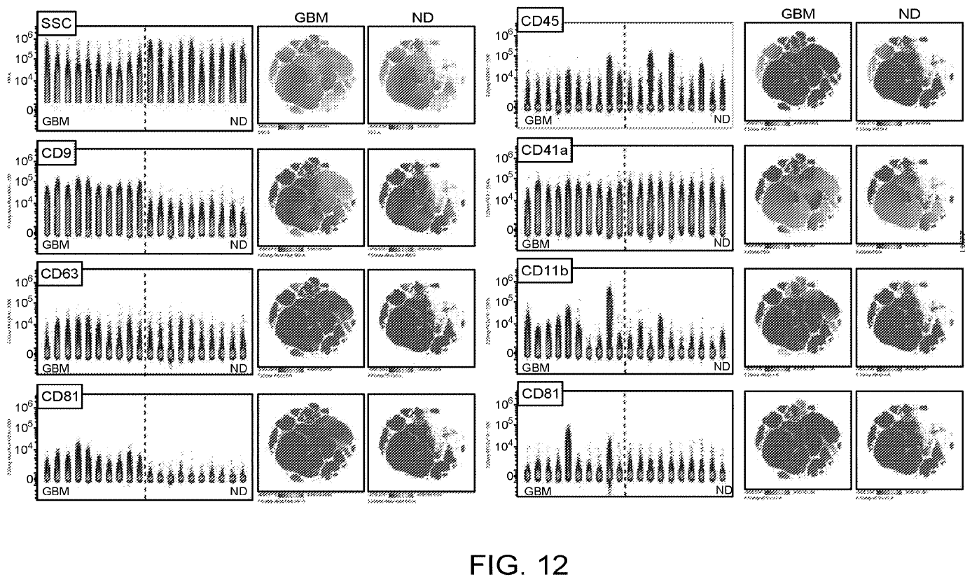

t-SNE analysis based on relative SSC, CD9, CD63, CD81, CD31, CD45, CD11b, and CD41a values on a data set combining 10 GBM patients and 10 normal donors (FIG. 5A) revealed distinct clusters enriched in GBM patients and normal donors. GBM patients' plasma EVs had increased CD9, CD81 and CD11b expression (FIG. 12). FlowSOM revealed 15 distinct EV subpopulations (FIGS. 5B-5C). Pop1 (mean 34.76% vs 0.52, p<0.0001), Pop3 (mean 6.34% vs 0.04%, p<0.0001), and Pop7 (mean 0.32% vs 0.001%, p=0.015) were enriched in GBM patients while Pop0 (mean 49.08% vs 76.31%, p<0.001), Pop2 (mean 0.09% vs 0.20%, p=0.016), and Pop6 (mean 2.16% vs 11.92%, p<0.001) were enriched in normal donors.

### Example 2: A Distinct CD9⁺ Plasma EV Phenotype in GBM Patients

Plasma EVs from 20 GBM patients and 20 brain metastases (mets) patients were analyzed with flow cytometry-based expression of phalloidin/actin, CD9, CD63, CD81, CD11b, CD45, CD31, CD41a, and light side scatter (SCC; a correlate of particle size).

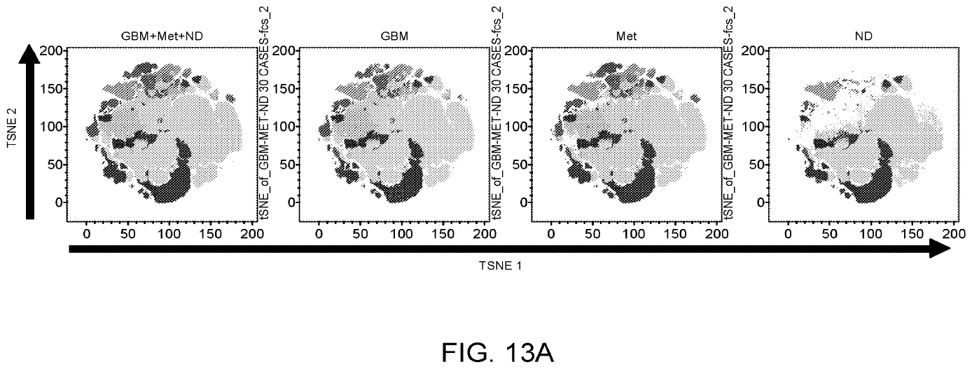

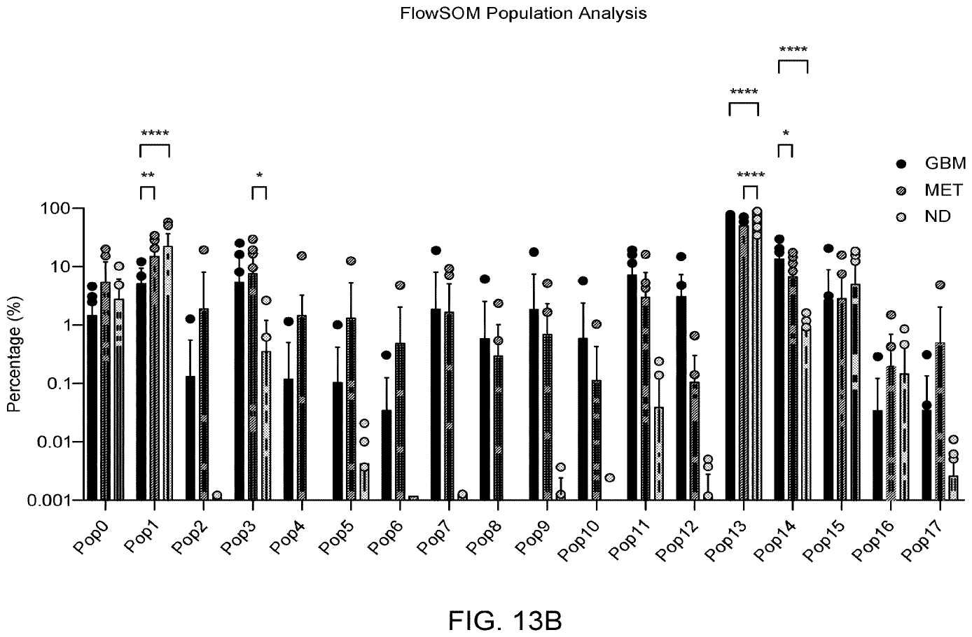

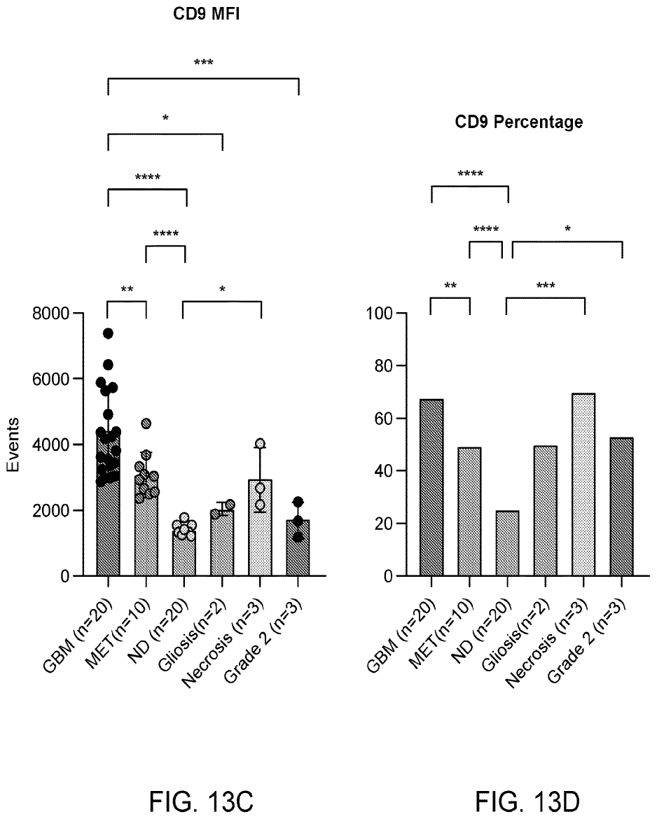

Flow cytometry and TSNE analysis distinguished samples from each other and from normal donors based on a novel surface markers and size (FIGS. 13A-13B). Furthermore, EV CD9 expression alone differentiated between GBM, brain mets, normal donors, and other brain lesions (FIG. 13C).

### Example 3: Distinct CD9⁺ Plasma EV Phenotype in GBM Patients Also can Distinguish Between Primary GBM and Metastatic GBM

Plasma EVs were assessed as described in Example 1.

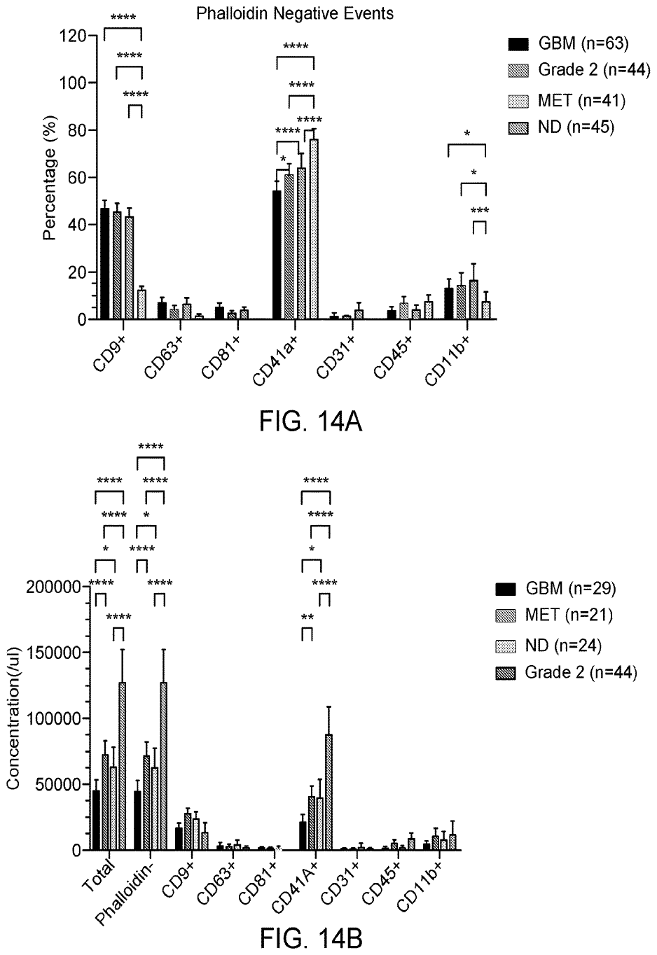

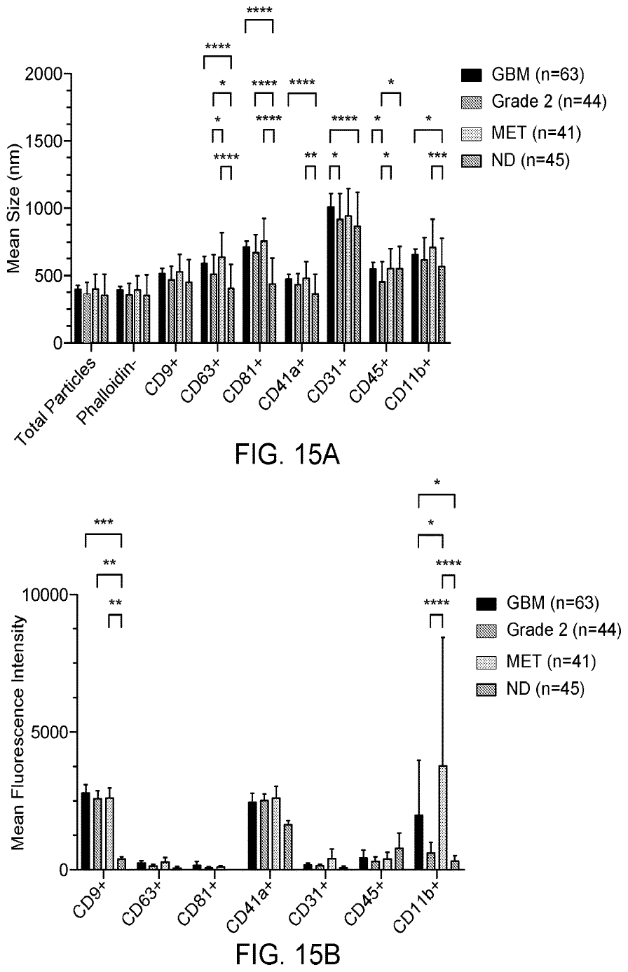

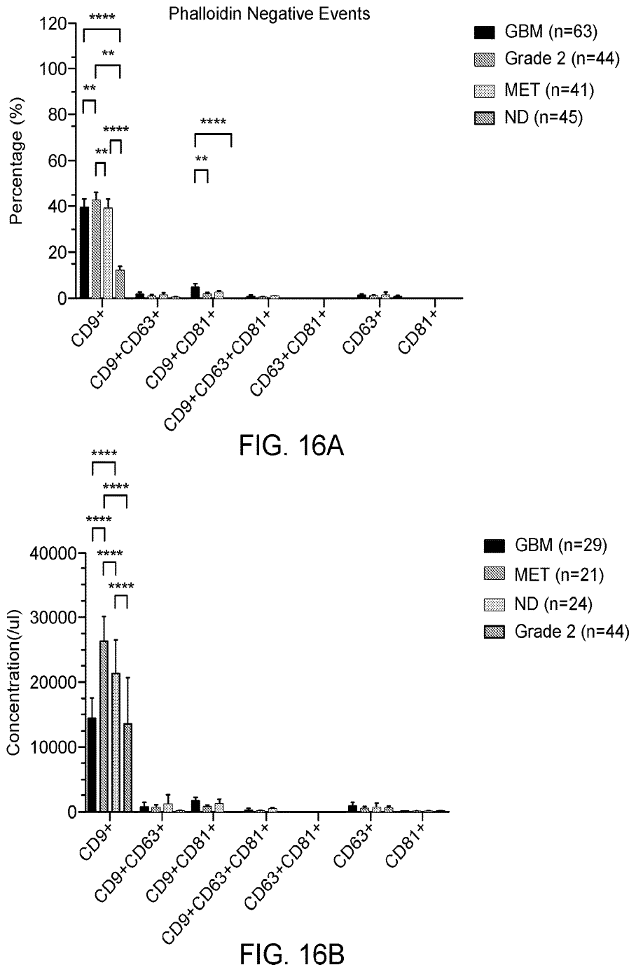

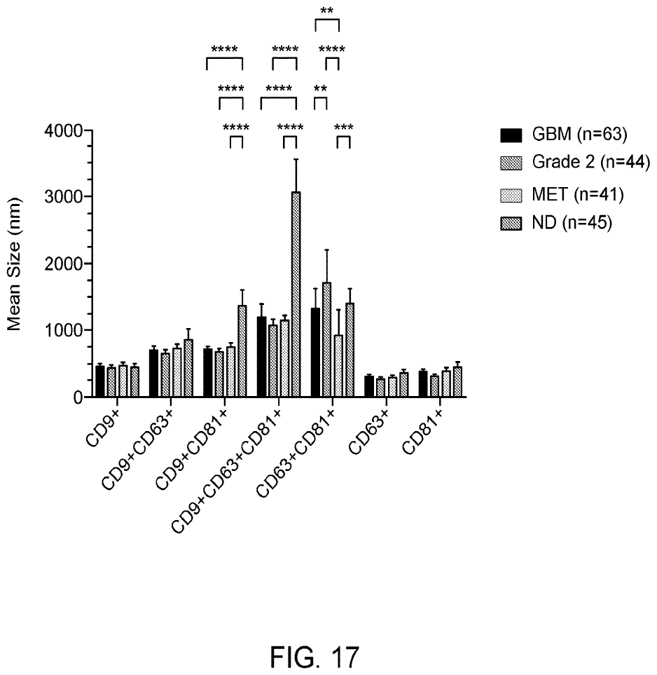

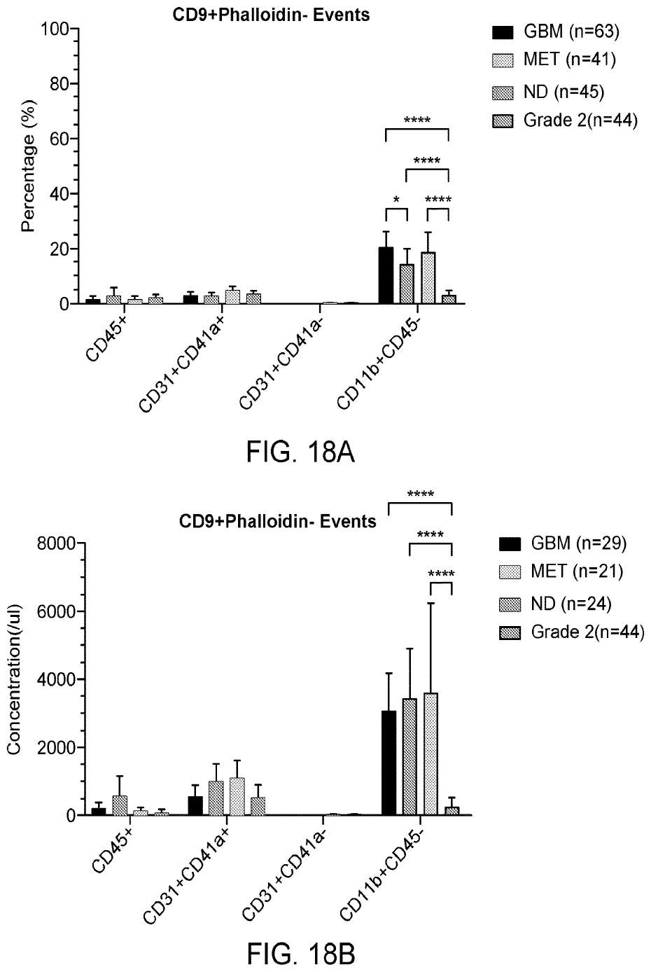

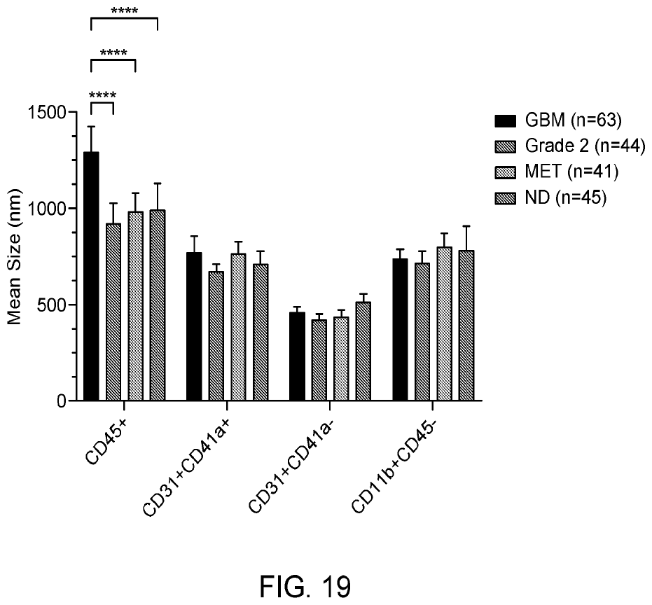

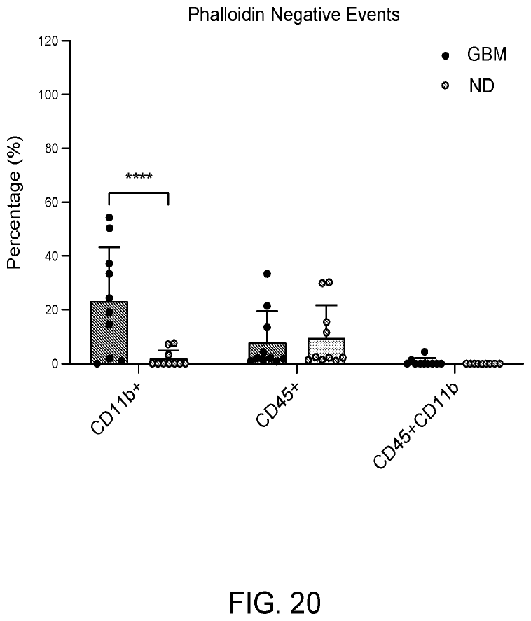

CD9+ EVs were more frequent as a percentage of plasma EVs in GBM and brain mets patients than in normal donors (FIG. 14A). CD9+ EVs were found at higher concentration in brain mets patients than GBM patients (FIG. 16B).

EV percentage, concentration, mean fluorescence intensity, size, and combinations thereof can be used to identify mammals (e.g., humans) as having GBM and in some cases can be used to classify the GBM as a primary GBM or a metastatic GBM.

All percentages and concentrations were based on a subset of particles already identified by gating on particles in the general EV size range.

### Phalloidin-Negative Particles.

Concentration:
   >75,000 events/μL=normal donor
   50,000-75,000 events/μL=grade 2 glioma and brain mets
   <50,000 events/μL=GBM

### Single Markers (Already Gated on Phalloidin Negative)

CD9
   Percentage
      >15%=GBM, grade 2 glioma, and brain mets
      <15%=normal donor
   Mean fluorescence intensity
      <1000=normal donor
      >1000=GBM, grade 2 glioma, brain mets
CD41a
   Percentage
      >75%=normal donor
      55%-75%=grade 2 glioma and brain mets
      <55%=GBM
   Concentration
      >50,000 events/μL=normal donor
      25,000-50,000 events/μL=grade 2 glioma and brain mets
      <25,000 events/ptL=GBM
CD11b
   Percentage:
      >10%=GBM, grade 2 glioma, and brain mets
      <10%=normal donor
   Mean fluorescence intensity
      <250=normal donor
      250-500=grade 2 glioma
      >500=GBM, brain mets

### Combinations of Tetraspanin Proteins (i.e., EV Markers CD9, CD63, CD81)

CD9⁺/CD81⁺
   Percentage
      >1%=GBM
      0.1%-1%=grade 2 glioma or brain mets
      <0.1%=normal donor
   Size
      >750 nm=normal donor
      <750 nm=GBM, grade 2 glioma, brain mets
CD9⁺/CD63⁺/CD81⁺
   Size
      >1400 nm=normal donor
      <1400 nm=GBM, grade 2 glioma, brain mets
         Combinations of Markers of Cell Origin Among CD9⁺/Phalloidin⁻ Particles
“CD11b⁺/CD45⁻
   Percentage
      >5%=GBM, grade 2 glioma, brain mets
      <5%=normal donor
   Concentration
      >500 events/μL=GBM, grade 2 glioma, brain mets
      <500 events/μL=normal donor
CD45⁺
   Size
      >1000 nm=GBM
      <1000 nm=grade 2 glioma, brain mets, normal donor

### Example 4: a Distinct CD9⁺ Plasma EV Phenotype in GBM Patients

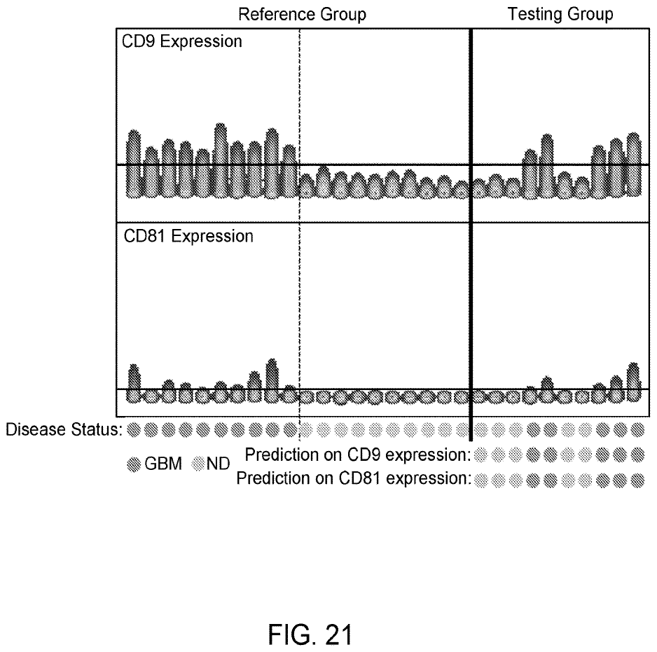

Plasma EVs from 5 GBM patients and 5 normal donors (ND) were analyzed with flow cytometry-based expression of CD9 and CD81. CD9 and CD81 expression thresholds were based on GBM and ND reference groups and the same thresholds were applied to the test group. Both CD9 and CD81 expression accurately distinguished GBM patients from ND (FIG. 21).

### Example 5: A Distinct CD9⁺ Plasma EV Phenotype in Pre- and Post-Op GBM Patients

Plasma EVs from 31 pre-operative GBM patients, 10 post-operative GBM patients, and 20 normal donors were analyzed with flow cytometry-based expression of phalloidin/actin, tetraspanins (e.g., CD9, CD63, and CD81), myeloid markers (e.g., CD11b), leukocyte markers (e.g., CD45), and platelet and endothelial cell markers (e.g., CD31 and CD41a).

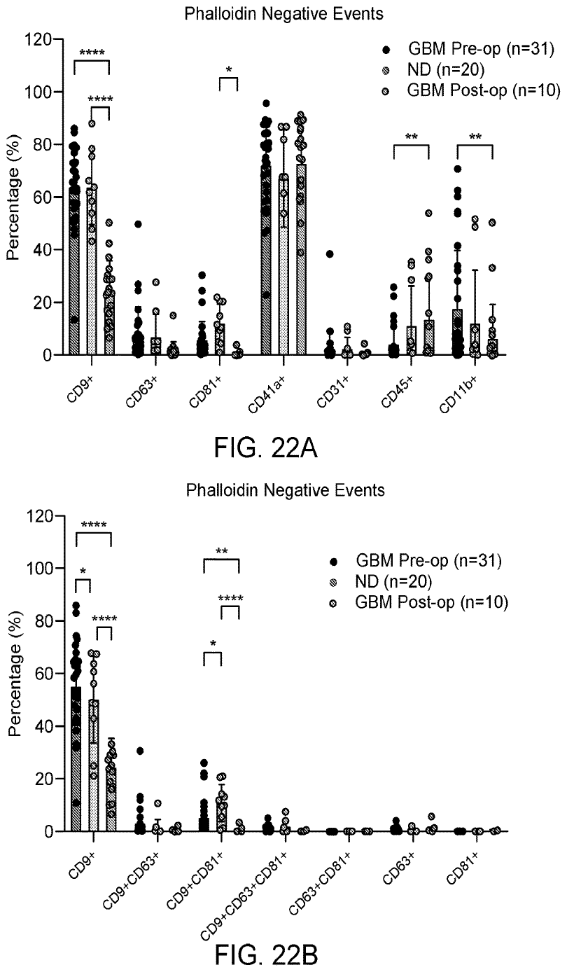

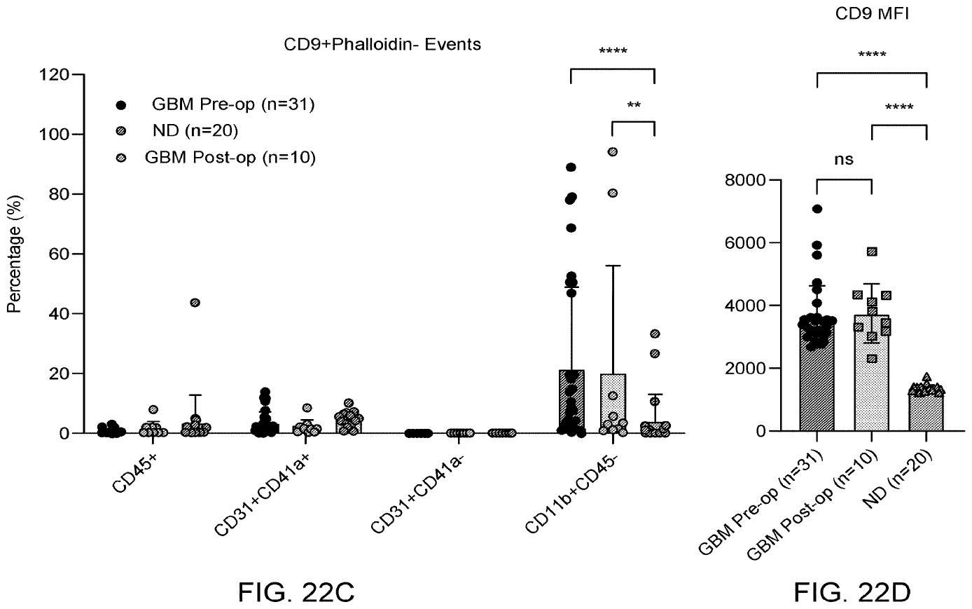

CD9+ plasma EVs expressing tetraspanins (CD9, CD63, CD81), leukocyte markers (CD45), platelet and endothelial cell markers (CD31, CD41a), and myeloid markers (CD11b) alone or in combination were present in varying frequencies (percentage) (FIGS. 22A-22C) and had distinct mean fluorescence intensities (MFI) (FIG. 22D) in pre-operative GBM patients, post-operative GBM patients, and normal donors. Pre-operative and post-operative GBM EVs showed differentiated CD9 and CD81 expression (FIG. 22B). Accordingly, the CD9⁺ plasma EV phenotype in GBM patients can be used to determine whether that patient has previously undergone surgery for that GBM. For example, the CD9⁺ plasma EV phenotype in GBM patients can be used to determine whether that patient has a pre-operative GBM or a post-operative GBM.

### Example 6: Plasma EV Phenotype Correlates with Tumor Volume and Overall Survival

Plasma EVs from GBM patients were analyzed with flow cytometry-based expression of phalloidin/actin, CD9, CD63, CD81, and CD41a at diagnosis.

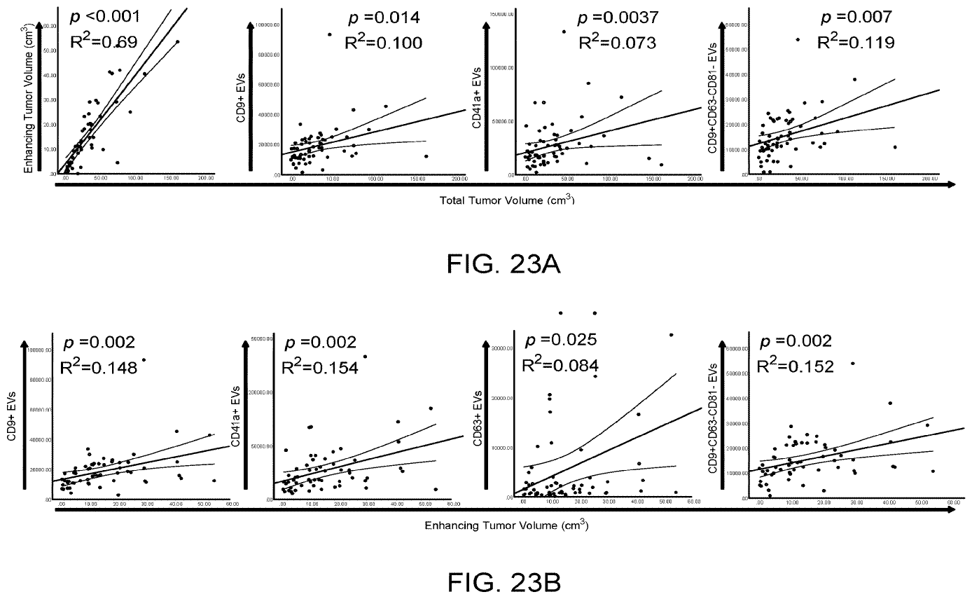

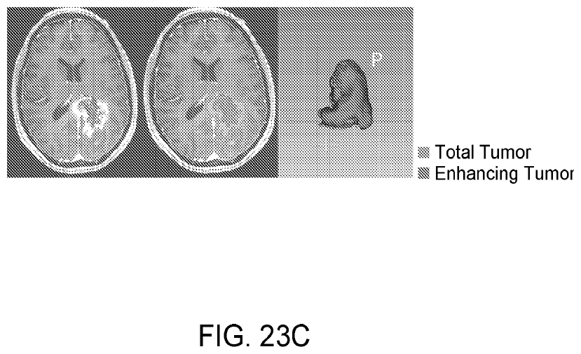

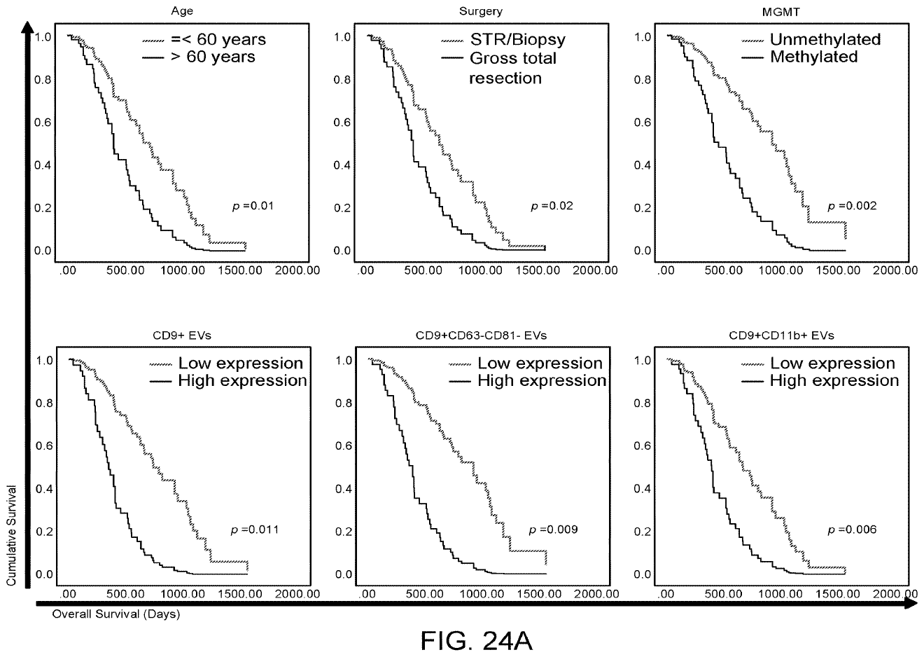

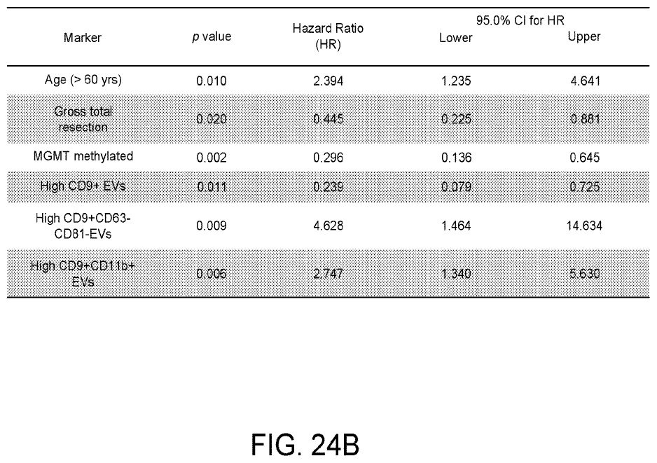

The frequency of CD9+, CD41a+, CD63+, and CD9+/CD63−/CD81− plasma EVs in GBM at diagnosis showed significant correlation with both total tumor volume (FIG. 23A) and enhancing tumor volume (FIG. 23B). Enhancing tumor volume can be used to guide surgical decision making (FIG. 23C). Furthermore, frequency of CD9+, CD9+/CD63−/CD81−, and CD9+/CD11b+ plasma EVs in GBM at diagnosis showed independent correlation with overall survival, along with age, gross total resection, and MGMT promoter methylation (FIG. 24A). Specifically, high CD9+ EVs (p value=0.011), high CD9+/CD63−/CD81− EVs (p value=0.009), and high CD9+/CD11b+ EVs (p value 0.006) were each significantly correlated with overall survival (FIG. 24B).

### Example 7: A Machine Learning Analysis of GBM Plasma EVs

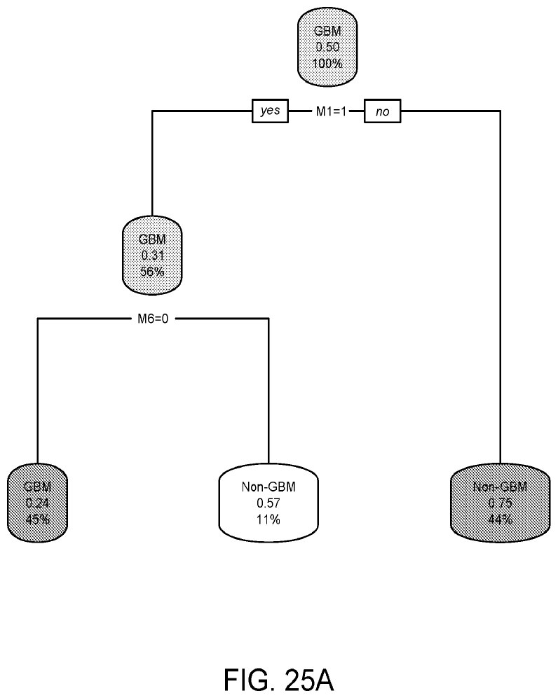

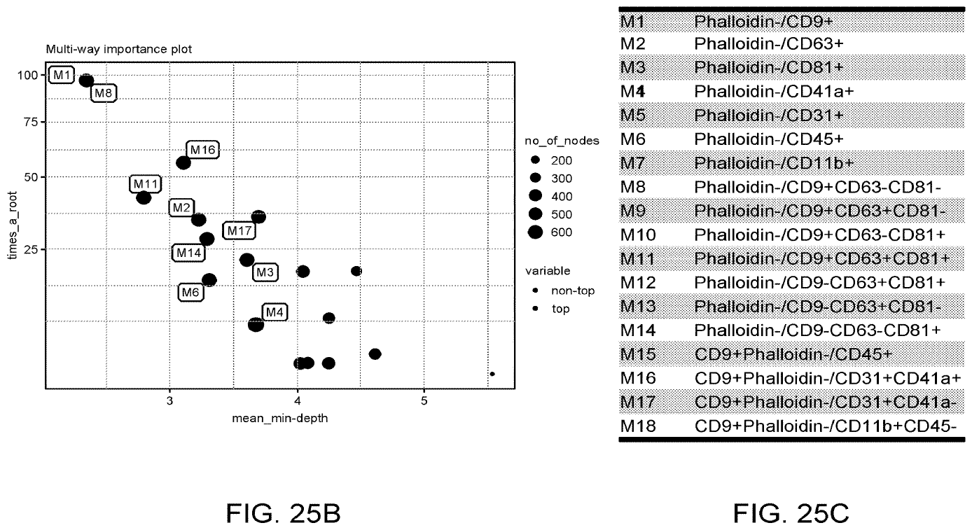

Plasma EVs from 40 GBM patients, 40 non-GBM patients (20 normal donors and 20 brain metastases (mets) patients) that demonstrated expression of phalloidin/actin, CD9, CD63, CD81, CD11b, CD45, CD31, and CD41a by flow cytometry were subjected to a machine learning analysis as shown in FIG. 25A.

Percentages of expression levels (continuous) were transformed into binary categories of high or low expression, using the mean expression level of all samples as a cutoff. These data were used to train and test a machine learning algorithm (80/20 data split-80% training set and 20% testing set). The resulting machine learning algorithm produced predictions of GBM with 85.71% sensitivity, 77.8% specificity, 75% positive predictive value (PPV), 87.5% negative predictive value (NPV), and 81.25% accuracy (FIGS. 25B and 25C).

### Example 8: Treating GBM

A blood sample (e.g., a plasma sample) is obtained from a human suspected of having GBM. CD9⁺ EVs in the obtained sample are examined to determine (a) whether the CD9⁺EVs contain at least one of (1) a CD63 polypeptide, (2) a CD81 polypeptide, (3) a CD11b polypeptide (4) a CD41a polypeptide, and (5) a CD45 polypeptide, and/or (b) the size of the CD9⁺ EVs.

If the CD9⁺ EVs in the sample (a) contain at least one of (1) a CD63 polypeptide, (2) a CD81 polypeptide, (3) a CD11b polypeptide, (4) a CD41a polypeptide, and (5) a CD45 polypeptide and/or (b) have a size (e.g., an average longest dimension such as an average diameter) that is different (e.g., as compared to CD9⁺ EVs from patients who do not have GBM), then the human is subjected to radiation therapy and is administered temozolomide. For example, if the CD9⁺ EVs in the sample (a) contain (e.g., on a surface of the vesicle) a CD81 polypeptide (e.g., greater than 1 percent of CD9⁺ EVs in the blood sample contain a CD81 polypeptide), and (b) have an average longest dimension (e.g., an average diameter) of less than about 750 nm, then the human is subjected to radiation therapy and is administered temozolomide. For example, if the CD9⁺ EVs in the sample (a) contain (e.g., on a surface of the vesicle) a CD63 polypeptide and a CD81 polypeptide, and (b) have an average longest dimension (e.g., an average diameter) of from less than about 1400 nm, then the human is subjected to radiation therapy and is administered temozolomide.

The radiation therapy and the administered temozolomide reduce the number of cancer cells within the human.

### Example 9: Treating Metastatic GBM

A blood sample (e.g., a plasma sample) is obtained from a human suspected of having GBM. CD9⁺ EVs in the obtained sample are examined to determine (a) whether the CD9⁺ EVs contain at least one of (1) a CD63 polypeptide, (2) a CD81 polypeptide, (3) a CD11b polypeptide, (4) a CD41a polypeptide, and (5) a CD45 polypeptide and/or (b) the size of the CD9⁺ EVs.

If the CD9⁺ EVs in the sample (a) contain at least one of (1) a CD63 polypeptide, (2) a CD81 polypeptide, (3) a CD11b polypeptide, (4) a CD41a polypeptide, and (5) a CD45 polypeptide and/or (b) have a size (e.g., an average longest dimension such as an average diameter) that is different (e.g., as compared to CD9⁺ EVs from patients who do not have GBM), then the human is subjected to radiation therapy and administered temozolomide. For example, if the CD9⁺ EVs in the sample (a) contain (e.g., on a surface of the vesicle) a CD81 polypeptide (e.g., greater than 1 percent of CD9⁺ EVs in the blood sample contain a CD81 polypeptide), and (b) have an average longest dimension (e.g., an average diameter) of less than about 750 nm, then the human is subjected to a radiosurgery such as stereotactic radiosurgery (e.g., and is not administered any temozolomide). For example, if the CD9⁺ EVs in the sample (a) contain (e.g., on a surface of the vesicle) a CD63 polypeptide and a CD81 polypeptide, and (b) have an average longest dimension (e.g., an average diameter) of from less than about 1400 nm, then the human is subjected to a radiosurgery such as stereotactic radiosurgery (e.g., and is not administered any temozolomide).

The radiosurgery reduces the number of cancer cells within the human.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-7. (canceled)

8. A method for treating brain cancer, wherein said method comprises:
(a1) determining that greater than 1 percent of CD9⁺ EVs in a blood sample obtained from a mammal suspected of having brain cancer contain a CD81 polypeptide,
(a2) determining that CD9⁺ EVs in a blood sample obtained from a mammal suspected of having brain cancer contain a CD81 polypeptide and have a size of less than 750 nm,
(a3) determining that greater than 5 percent of CD9⁺ EVs in a blood sample obtained from a mammal suspected of having brain cancer contain a CD11b polypeptide,
(a4) determining that CD9⁺ EVs containing a CD63 polypeptide and a CD81 polypeptide in a blood sample obtained from a mammal suspected of having brain cancer have a size of less than 1400 nm,
(a5) determining that CD9⁺ EVs containing a CD11b polypeptide in a blood sample obtained from a mammal suspected of having brain cancer have a concentration of greater than 500 events/μL, or
(a6) determining that CD9⁺ EVs containing a CD45 polypeptide in a blood sample obtained from a mammal suspected of having brain cancer have a size of greater than 1000 nm, and
(b) administering a brain cancer treatment to said mammal.

9. The method of claim 8, wherein said mammal is a human.

10. The method of claim 8, wherein said brain cancer is selected from the group consisting of a glioma, an acoustic neuroma, a pituitary adenoma, and a medulloblastoma.

11. The method of claim 10, wherein the glioma is a glioblastoma.

12. The method of claim 9, wherein said blood sample is selected from the group consisting of a whole blood sample, a plasma sample, and a serum sample.

13. The method of claim 9, wherein said brain cancer treatment comprises subjecting the mammal to radiation therapy.

14. The method of claim 9, wherein said brain cancer treatment comprises administering temozolomide to said mammal.

15. The method of claim 9, wherein said brain cancer treatment comprises subjecting the mammal to radiation therapy and administering temozolomide to said mammal.

16. A method for treating brain cancer, wherein said method comprises administering a brain cancer treatment to a mammal identified as having a blood sample comprising (i) greater than 1% of CD9⁺ EVs in said blood sample that contain a CD81 polypeptide, (ii) CD9⁺ EVs that contain a CD81 polypeptide and have a size of less than 750 nm, (iii) greater than 5% of CD9⁺ EVs in said blood sample that contain a CD11b polypeptide, (iv) CD9⁺ EVs that contain a CD63 polypeptide and a CD81 polypeptide and have a size of less than 1400 nm, (v) a concentration of CD9⁺ EVs containing a CD11b polypeptide of greater than 500 events/μL, or (vi) CD9⁺ EVs containing a CD45 polypeptide and having a size of greater than 1000 nm.

17. The method of claim 16, wherein said mammal is a human.

18. The method of claim 16, wherein said brain cancer is selected from the group consisting of a glioma, an acoustic neuroma, a pituitary adenoma, and a medulloblastoma.

19. The method of claim 18, wherein the glioma is a glioblastoma.

20. The method of claim 16, wherein said blood sample is selected from the group consisting of a whole blood sample, a plasma sample, and a serum sample.

21. The method of claim 16, wherein said brain cancer treatment comprises subjecting the mammal to radiation therapy.

22. The method of claim 16, wherein said brain cancer treatment comprises administering temozolomide to said mammal.

23. The method of claim 16, wherein said brain cancer treatment comprises subjecting the mammal to radiation therapy and administering temozolomide to said mammal.

24-123. (canceled)

124. The method of claim 8, wherein said method comprises (a1).

125. The method of claim 8, wherein said method comprises (a2).

126. The method of claim 16, wherein said blood sample comprises (i).

127. The method of claim 16, wherein said blood sample comprises (ii).
